SEQUENCE LISTING (7 sequences)

SEQ ID NO: 1 - protein, 149 aa, homo sapiens
  misc_feature: PlGF-1, 149 aa, n P49763-2
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERCGDAVPRR

SEQ ID NO: 2 - protein, 170 aa, homo sapiens
  misc_feature: PlGF-2, 170aa, n P49763-3
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 3 - protein, 221 aa, homo sapiens
  misc_feature: PlGF-3, 221 aa, n P49763-1
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRHSPGRQSPDMPGDFRADAPSFLPPRRSLPMLFRMEWGCALTGSQSAVWPSSPVPEEIPRMHPGRNGKKQQRKPLREKMKPERCGDAVPRR

SEQ ID NO: 4 - protein, 242 aa, homo sapiens
  misc_feature: PlGF-4, 242 aa, n P49763-4
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRHSPGRQSPDMPGDFRADAPSFLPPRRSLPMLFRMEWGCALTGSQSAVWPSSPVPEEIPRMHPGRNGKKQQRKPLREKMKPERRRPKGRGKRRREKQRPTDCHLCGDAVPRR

SEQ ID NO: 5 - DNA, 1911 bp, Homo sapiens
  misc_feature: mRNA of PlGF-1,  (NCBI  NM_002632.5)
cctcgcacgcactgcgggctccggcgctgcgggctggccgggcgctgcgggctgaccgggcgctccgggaactcggctcgggaacctcgtctgcggtgggcggggccggcccggagccccgccccggctcagtccctgaaacccaggcgcggaccggctgcagtctcagaagggagctgctgtctgcggaggaaactgcatcgacggacggccgcccagctacgggaggacctggagtggcactgggcgcccgacggaccatccccgggacccgcctgcccctcggcgccccgccccgccgggccgctccccgtcgggttccccagccacagccttacctacgggctcctgactccgcaaggcttccagaagatgctcgaaccaccggccggggcctcggggcagcagtgagggaggcgtccagccccccactcagctcttctcctcctgtgccaggggctccccgggggatgagcatggtggttttccctcggagccccctggctcgggacgtctgagaagatgccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggaggagacccaagggcagggggaagaggaggagagagaagcagagacccacagactgccacctgtgcggcgatgctgttccccggaggtaacccaccccttggaggagagagaccccgcacccggctcgtgtatttattaccgtcacactcttcagtgactcctgctggtacctgccctctatttattagccaactgtttccctgctgaatgcctcgctcccttcaagacgaggggcagggaaggacaggaccctcaggaattcagtgccttcaacaacgtgagagaaagagagaagccagccacagacccctgggagcttccgctttgaaagaagcaagacacgtggcctcgtgaggggcaagctaggccccagaggccctggaggtctccaggggcctgcagaaggaaagaagggggccctgctacctgttcttgggcctcaggctctgcacagacaagcagcccttgctttcggagctcctgtccaaagtagggatgcggatcctgctggggccgccacggcctggctggtgggaaggccggcagcgggcggaggggatccagccacttccccctcttcttctgaagatcagaacattcagctctggagaacagtggttgcctgggggcttttgccactccttgtcccccgtgatctcccctcacactttgccatttgcttgtactgggacattgttctttccggccaaggtgccaccaccctgccccccctaagagacacatacagagtgggccccgggctggagaaagagctgcctggatgagaaacagctcagccagtggggatgaggtcaccaggggaggagcctgtgcgtcccagctgaaggcagtggcaggggagcaggttccccaagggccctggcacccccacaagctgtccctgcagggccatctgactgccaagccagattctcttgaataaagtattctagtgtggaaacgct

SEQ ID NO: 6 - DNA, 1848 bp, Homo sapiens
  misc_feature: mRNA of PlGF-2, (NCBI NM_001207012.1)
cctcgcacgcactgcgggctccggcgctgcgggctggccgggcgctgcgggctgaccgggcgctccgggaactcggctcgggaacctcgtctgcggtgggcggggccggcccggagccccgccccggctcagtccctgaaacccaggcgcggaccggctgcagtctcagaagggagctgctgtctgcggaggaaactgcatcgacggacggccgcccagctacgggaggacctggagtggcactgggcgcccgacggaccatccccgggacccgcctgcccctcggcgccccgccccgccgggccgctccccgtcgggttccccagccacagccttacctacgggctcctgactccgcaaggcttccagaagatgctcgaaccaccggccggggcctcggggcagcagtgagggaggcgtccagccccccactcagctcttctcctcctgtgccaggggctccccgggggatgagcatggtggttttccctcggagccccctggctcgggacgtctgagaagatgccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggtgcggcgatgctgttccccggaggtaacccaccccttggaggagagagaccccgcacccggctcgtgtatttattaccgtcacactcttcagtgactcctgctggtacctgccctctatttattagccaactgtttccctgctgaatgcctcgctcccttcaagacgaggggcagggaaggacaggaccctcaggaattcagtgccttcaacaacgtgagagaaagagagaagccagccacagacccctgggagcttccgctttgaaagaagcaagacacgtggcctcgtgaggggcaagctaggccccagaggccctggaggtctccaggggcctgcagaaggaaagaagggggccctgctacctgttcttgggcctcaggctctgcacagacaagcagcccttgctttcggagctcctgtccaaagtagggatgcggatcctgctggggccgccacggcctggctggtgggaaggccggcagcgggcggaggggatccagccacttccccctcttcttctgaagatcagaacattcagctctggagaacagtggttgcctgggggcttttgccactccttgtcccccgtgatctcccctcacactttgccatttgcttgtactgggacattgttctttccggccaaggtgccaccaccctgccccccctaagagacacatacagagtgggccccgggctggagaaagagctgcctggatgagaaacagctcagccagtggggatgaggtcaccaggggaggagcctgtgcgtcccagctgaaggcagtggcaggggagcaggttccccaagggccctggcacccccacaagctgtccctgcagggccatctgactgccaagccagattctcttgaataaagtattctagtgtggaaacgct

SEQ ID NO: 7 - DNA, 1908 bp, Homo sapiens
  misc_feature: mRNA of PlGF-3, (NCBI  NM_001293643.1)
cctcgcacgcactgcgggctccggcgctgcgggctggccgggcgctgcgggctgaccgggcgctccgggaactcggctcgggaacctcgtctgcggtgggcggggccggcccggagccccgccccggctcagtccctgaaacccaggcgcggaccggctgcagtctcagaagggagctgctgtctgcggaggaaactgcatcgacggacggccgcccagctacgggaggacctggagtggcactgggcgcccgacggaccatccccgggacccgcctgcccctcggcgccccgccccgccgggccgctccccgtcgggttccccagccacagccttacctacgggctcctgactccgcaaggcttccagaagatgctcgaaccaccggccggggcctcggggcagcagtgagggaggcgtccagccccccactcagctcttctcctcctgtgccaggggctccccgggggatgagcatggtggttttccctcggagccccctggctcgggacgtctgagaagatgccggtcatgaggctgttcccttgcttcctgcagctcctggccgggctggcgctgcctgctgtgcccccccagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggaggagacccaagggcagggggaagaggaggagagagaagcagagacccacagactgccacctgtgcggcgatgctgttccccggaggtaacccaccccttggaggagagagaccccgcacccggctcgtgtatttattaccgtcacactcttcagtgactcctgctggtacctgccctctatttattagccaactgtttccctgctgaatgcctcgctcccttcaagacgaggggcagggaaggacaggaccctcaggaattcagtgccttcaacaacgtgagagaaagagagaagccagccacagacccctgggagcttccgctttgaaagaagcaagacacgtggcctcgtgaggggcaagctaggccccagaggccctggaggtctccaggggcctgcagaaggaaagaagggggccctgctacctgttcttgggcctcaggctctgcacagacaagcagcccttgctttcggagctcctgtccaaagtagggatgcggatcctgctggggccgccacggcctggctggtgggaaggccggcagcgggcggaggggatccagccacttccccctcttcttctgaagatcagaacattcagctctggagaacagtggttgcctgggggcttttgccactccttgtcccccgtgatctcccctcacactttgccatttgcttgtactgggacattgttctttccggccaaggtgccaccaccctgccccccctaagagacacatacagagtgggccccgggctggagaaagagctgcctggatgagaaacagctcagccagtggggatgaggtcaccaggggaggagcctgtgcgtcccagctgaaggcagtggcaggggagcaggttccccaagggccctggcacccccacaagctgtccctgcagggccatctgactgccaagccagattctcttgaataaagtattctagtgtggaaacgct

(19) United States
(12) Patent Application Publication
(10) Pub. No.: US 2019/0351018 A1
(43) Pub. Date: Nov. 21, 2019

(54) PLACENTAL GROWTH FACTOR FOR THE TREATMENT OF FETAL ALCOHOL SYNDROME DISORDERS (FASD)

(71) Applicants: UNIVERSITE DE ROUEN NORMANDIE, MONT SAINT AIGNAN (FR); CENTRE HOSPITALIER UNIVERSITAIRE DE ROUEN, ROUEN (FR); INSTITUT NATIONAL DE LA SANTE ET DE LA RECHERCHE MEDICALE (INSERM), PARIS (FR)

(72) Inventors: Bruno José GONZALEZ, LA VAUPALIERE (FR); Stéphane MARRET, ROUEN (FR); Matthieu Jean Alexandre LECUYER, ROUEN (FR); Annie LAQUERRIERE, BONSECOURS (FR); Soumeya BEKRI, ST LEGER DU BOURG DENIS (FR); Céline LESUEUR, MONT SAINT AIGNAN (FR); Sylvie Marguerite Alberte JEGOU, ROUEN (FR); Pascale Yvonne Joséphine MARCORELLES, BREST (FR)

(73) Assignees: UNIVERSITE DE ROUEN NORMANDIE, MONT SAINT AIGNAN (FR); CENTRE HOSPITALIER UNIVERSITAIRE DE ROUEN, ROUEN (FR); INSTITUT NATIONAL DE LA SANTE ET DE LA RECHERCHE MEDICALE (INSERM), PARIS (FR)

(21) Appl. No.: 16/465,465

(22) PCT Filed: Dec. 1, 2017
(86) PCT No.: PCT/EP2017/081159

§ 371 Date: May 30, 2019

(30) Foreign Application Priority Data
Dec. 1, 2016 (FR) 1661813

(51) Int. Cl. A61K 38/18; C12Q 1/6883

(52) U.S. Cl. CPC A61K 38/1866; C12Q 1/6883; C12Q 2600/112; C12Q 2600/118; C12Q 2600/158

(57) ABSTRACT

The invention relates to a placental growth factor (PlGF) to be used as a drug in the prevention and/or treatment of fetal alcohol syndrome disorders (FASD) selected from the group comprising fetal alcohol syndrome (FAS), cerebrovascular disease, and growth retardation in a subject exposed to alcohol in utero. The invention also relates to a pharmaceutical composition or a product comprising the PlGF for the same therapeutic uses.

Alcohol is a teratogen responsible for physical and behavioral disorders. In humans, prenatal alcohol exposure can lead to alterations in brain development. Alcohol consumption during pregnancy (fetal alcohol exposure), for example, is the leading cause of disability and in particular of mental retardation of nongenetic origin in France and worldwide.

Damage varies according to the period when the fetus was exposed, blood alcohol levels, genetic and environmental factors, and consumption patterns (chronic, binge drinking).

Fetal alcohol syndrome (FAS) is the most extreme and disabling fetal alcohol spectrum disorder (FASD). Its incidence is estimated in France at 1.5% of births. FAS combines physical abnormalities such as hypotrophy (growth retardation), craniofacial dysmorphism and neurobehavioral abnormalities resulting in cognitive function disorders (attention, motor, learning or memory disorders). Neurological sequelae are also present in children with FASD, the incidence of which is estimated in France at nearly 1% of births.

Brain angiogenesis is concomitant with the neurogenesis process and contributes to good brain development by providing nerve cells with nutrients, oxygen and trophic factors. In particular, brain angiogenesis is a prerequisite for the proper development of the neuronal network. In addition, a direct impact of brain vessels on the migration process of neuronal populations and oligodendrocytes has recently been demonstrated. It has also been previously established by the inventors that in utero alcohol exposure disrupts brain angiogenesis and that this effect contributes to the brain abnormalities of alcohol exposure.

Targeting vascular abnormalities of in utero alcohol exposure thus appears to be a therapeutic strategy to reduce the neurodevelopmental damage of alcohol and correct impaired brain function. Indeed, the current management of children with FASD aims, through stimulation of motor and cognitive functions, to reduce language, learning or attention disorders that are often diagnosed late, often between 4 and 5 years of age, during schooling. The results obtained by such follow-up are limited and in utero alcohol exposure disorders will have a long-term impact on the social and professional integration of these future adults. There is today no curative treatment for the neurodevelopmental effects of alcohol in children with FASD.

There is therefore a need to develop a new therapy for disorders related to in utero alcohol exposure intended to treat in particular abnormal brain vascularization, to improve brain angiogenesis or the associated developmental disorders.

### DESCRIPTION

The inventors have previously demonstrated that the determination of placental growth factor (PlGF) in the placenta can identify, among children exposed to alcohol in utero, those who have suffered brain damage. In particular, these children have a decrease in placental PlGF levels corresponding to impaired brain angiogenesis.

Subsequently, in a preclinical model of in utero alcohol exposure, the inventors discovered that placental PlGF supplementation corrects the action of alcohol on morphometric and anatomical parameters indicative of fetal growth, such as body and head size at birth. In addition, the inventors have demonstrated that increasing the amount of PlGF strongly reduces the harmful effect of alcohol on fetal brain vascularization. In particular, it improves brain angiogenesis and corrects alcohol-induced microvessel disorganization.

Fetal growth factor (PlGF) has been described in the prior art as a target for the treatment of cardiovascular diseases, retinopathies, skin diseases and cancers (Lu et al., Discov Med, 2016 21:349-61, Aprile et al., Crit Rev Oncol Hematol, 2015 95:165-78 and Shibuya, Endocr Metab Immune Disord Drug Targets, 2015, 15:135-44). More specifically, PlGF has been described in the treatment of retinopathies in adults (US2015013359) and children (Hard et al., Fondation Act Paediatrica 2011 100, pp. 1063-1065). However, there are no studies suggesting the therapeutic effect of PlGF in the treatment of fetal alcohol spectrum disorders (FASD). In addition, treatments to correct the adverse effect of alcohol on brain vascularization and the growth of a subject exposed to alcohol in utero have not been identified. The present invention thus opens a new avenue in the prevention and/or treatment of FASD and, in particular, in the prevention and/or treatment of neurological disorders (such as hyperactivity, inattention, depression, anxiety, emotional disorders, excessive irritability, behavioral problems, etc.) due to abnormal vascular-dependent neuronal and oligodendrocyte migration in the brain or growth disorders (hypotrophy) following alcohol consumption during pregnancy.

In a first aspect, the present invention thus concerns a placental growth factor (PlGF) for use in the prevention and/or treatment of fetal alcohol spectrum disorders (FASD) in a subject who has been exposed to alcohol in utero.

The term “PlGF” or “placental growth factor” (these terms are synonymous) refers to a protein from the family of vascular endothelial growth factors (VEGF). In particular, PlGF within the meaning of the invention is a 149 amino acid protein highly similar to VEGF-A which is recognized by the same receptor as the latter, VEGF-R1, but which is not recognized by VEGF-R2. PlGF is strongly expressed by the placenta, but not by the fetus and especially by the fetal brain. N-terminal-glycosylated PlGF is secreted and functions as a dimer to control angiogenesis. The term “PlGF” refers in particular to all 4 isoforms, PlGF1-4: PlGF-1 and PlGF-3 are non-heparin-binding isoforms while PlGF-2 and PlGF-4 contain additional heparin-binding domains. Even more preferably, PlGF refers to a human protein whose sequence is selected from any one of the isoforms identified by UniProt accession numbers P49763-2 (PlGF-1 with amino acid sequences corresponding to SEQ ID NO: 1); P49763-3 (PlGF-2 with amino acid sequences corresponding to SEQ ID NO: 2); P49763-1 (PlGF-3 with amino acid sequences corresponding to SEQ ID NO: 3); P49763-4 (PlGF-4 with amino acid sequences corresponding to SEQ ID NO: 4) presented in the sequence listing attached to the present application.

According to one embodiment of the present invention, recombinant PlGF or protein analogues of human PlGF may also be used in the prevention and/or treatment of FASD.

In particular, the PlGF of the present invention is obtained by using a prokaryotic or eukaryotic recombinant protein production system, in particular by (i) culturing a microorganism or eukaryotic cells transformed using a nucleotide sequence encoding human PlGF (NCBI accession number of the gene 5228; accession number of the transcripts NM_002632.5 (SEQ ID NO: 5), NM001207012.1 (SEQ ID NO: 6), NM_001293643.1 (SEQ ID NO: 7)) and ii) isolating the protein produced by said microorganism or said eukaryotic cells. This technique is well-known to the skilled person. For more details, refer to the following book: Recombinant DNA Technology I, Editors Ales Prokop, Raskesh K Bajpai; Annals of the New York Academy of Sciences, Volume 646, 1991. The PlGF protein is preferably purified/isolated from cell lysates and/or from cell supernatants by which it is expressed and/or secreted. This purification may be carried out by any means known to the skilled person. A number of purification techniques are described in Voet D and Voet J G, Techniques of protein and nucleic acid purification.

Recombinant protein production systems use nucleotide vectors comprising nucleic acids encoding the polypeptides to be synthesized, which are introduced into host cells that produce said polypeptides (for more details, refer to “Recombinant DNA Technology I”, Editors Ales Prokop, Raskesh K Bajpai; Annals of the New York Academy of Sciences, Volume 646, 1991).

Many vectors into which a nucleic acid molecule of interest can be inserted in order to introduce and maintain it in a eukaryotic or prokaryotic host cell are known; the choice of an appropriate vector depends on the intended use of this vector (for example replication of the sequence of interest, expression of this sequence, maintenance of this sequence in extrachromosomal form or integration into the host's chromosomal material), as well as the nature of the host cell (for example, plasmids are preferably introduced into bacterial cells, while YACs are preferably used in yeasts). These expression vectors may be plasmids, YACs, cosmids, retroviruses, EBV-derived episomes, and any vectors that the skilled person may consider appropriate for the expression of said chains.

The vectors according to the invention comprise the nucleic acid encoding PlGF, or a similar sequence, as well as the means necessary for its expression. The expression “means necessary for the expression of a peptide”—the term peptide being used for any peptide molecule, such as protein, polyprotein, polypeptide, etc.—refers to any means by which the peptide can be obtained, such as in particular a promoter, a transcription terminator, an origin of replication and preferably a selectable marker. The means necessary for the expression of a peptide are operably linked to the nucleic acid sequence encoding the polypeptide fragment of the invention. “Operably linked” means a juxtaposition of said elements necessary for the expression of the gene encoding the polypeptide fragment of the invention, which are in a relationship such that they can function as expected. For example, there may be additional bases between the promoter and the gene encoding the polypeptide fragment of the invention as long as their functional relationship is preserved. The means necessary for the expression of a peptide may be homologous means, meaning naturally contained in the genome of the vector used, or they may be heterologous, meaning artificially added from another vector and/or organism. In the latter case, said means are cloned with the polypeptide fragment to be expressed. Examples of heterologous promoters include viral promoters such as the simian virus 40 (SV40) promoter, the herpes simplex virus thymidine kinase (TK-HSV-1) gene promoter, the Rous sarcoma virus (RSV) LTR, the cytomegalovirus (CMV) immediate-early promoter and the adenovirus major-late promoter (MLP), as well as any cell promoter that controls the transcription of genes encoding peptides in higher eukaryotes, such as the constitutive phosphoglycerate-kinase (PGK) gene promoter (Adra et al., Gene Volume 60, Issue 1, 1987, Pages 65-74), the promoter liver-specific genes alphal-antitrypsin and FIX and the SM22 promoter specific to smooth muscle cells (Moessler et al., Development 1996 August; 122(8):2415-25). The methods for deletion and insertion of DNA sequences in expression vectors are widely known to the skilled person and consist in particular of enzymatic digestion and ligation steps. The vectors of the invention may also include sequences necessary for targeting peptides to specific cell compartments. An example of targeting can be targeting to the endoplasmic reticulum obtained by using addressing sequences of the adenovirus E3 leader sequence type (Ciernik I. F., et al., The Journal of Immunology, vol. 162, 1999, pages 3915-3925).

The term “transcription terminator” refers here to a genome sequence that marks the end of the transcription of a gene or operon into messenger RNA. The transcription termination mechanism is different in prokaryotes and eukaryotes. The skilled person knows which signals to use according to the different cell types. For example, if the cell into which the vector is to be introduced is a bacterium, he or she will use a Rho-independent terminator (inverted repeat sequence followed by a series of T (uracils on the transcribed RNA) or a Rho-dependent terminator (consisting of a consensus sequence recognized by the Rho protein).

The term “origin of replication” (also called ori) is a unique DNA sequence that allows the initiation of replication. It is from this sequence that unidirectional or bidirectional replication begins. The skilled person knows that the structure of the origin of replication varies from one species to another; it is therefore specific to a species although it has some common characteristics between species. A protein complex is formed at this sequence and allows the DNA to be opened and replication to begin.

The vectors comprising the genetic sequence encoding PlGF are prepared by methods commonly used by the skilled person. The resulting clones can be introduced into an appropriate host by standard methods known to the skilled person to introduce polynucleotides into a host cell. Such methods may include dextran transformation, calcium phosphate precipitation, polybrene transfection, protoplast fusion, electroporation, polynucleotide encapsulation in liposomes, biolistic injection and direct DNA microinjection into the nucleus. Said sequence (isolated or inserted in a plasmid vector) can also be associated with a substance allowing it to cross the host cell membrane, for example a transporter such as a nanotransporter or a liposome preparation, or cationic polymers. In addition, these methods can be advantageously combined, for example by using electroporation with liposomes.

Examples of microorganisms suitable for the purposes of the invention include yeasts (Buckholz R G, Current Opinion in Biotechnology vol. 4, no. 5, 1993, pages 538-42) and bacteria (Olins and Lee, Current Opinion in Biotechnology vol. 4, no. 5, 1993, pages 520-5). Examples of eukaryotic cells include cells from animals such as mammals (Edwards C P and Aruffo A, Current Opinion in Biotechnology vol. 4, no. 5, 1993, pages 558-63), reptiles, and equivalent. Plant cells can also be used. The mammalian cells that can be used include Chinese hamster ovary (CHO) cells, monkey (COS and Vero) cells, baby hamster kidney (BHK) cells, pig kidney (PK 15) cells and rabbit kidney (RK13 cells, human osteosarcoma (143 B) cell lines, human HeLa cell lines and human hepatoma cell lines (Hep G2 cell type). It is also possible to use insect cells in which baculovirus processes can be used, for example (Luckow V A, Journal of Virology vol. 67, no. 8, 1993, pages 4566J9). In a preferred embodiment of the invention, the host cell used to produce the fragment of the invention is a bacterium, preferably E. coli.

The skilled person knows the conditions under which these cells are grown, as well as the experimental conditions necessary for the expression of polypeptide fragments by these cells.

The recombinant PlGF production process may include the following steps:
a) culture in a suitable medium and culture conditions of a host cell comprising the vector encoding PlGF; and
b) isolation of the PlGF produced in step a).

PlGF can be isolated (purified) from the cell in which it is expressed. In this case, a preliminary step of lysis of said cells may be necessary.

The culture media and conditions associated with each cell type used for the production of recombinant proteins are well-known to the skilled person.

The isolation (or purification) of PlGF can be done by any means known to the skilled person. Examples include differential precipitation or ultracentrifugation. It may also be advantageous to purify PlGF by ion-exchange chromatography, affinity chromatography, molecular sieving, or isoelectric focusing. All these techniques are described in Voet D and Voet J G, Techniques of Protein and Nucleic Acid Purification, Chapter 6, Biochemistry, 2nd edition.

More precisely, in a first step, the material from which the protein is to be extracted (animal or plant tissue, bacteria, etc.) is generally ground. Various devices (Waring blender, Potter-Eveljhem device, “Polytron”, etc.) can be used for this purpose. This homogenization is done in a buffer of suitable composition, well-known to the skilled person. The homogenate thus obtained is then clarified, most often by centrifugation, to remove large, poorly ground particles or to obtain the cell fraction containing the desired protein. If the protein is precisely in a cell compartment, a mild detergent (Triton X-100, Tween 20, sodium deoxycholate, etc.) is usually used to release it by dissolving the membranes of this compartment. The use of detergents must often be done in a controlled manner because they can rupture lysosomes, thus releasing hydrolytic enzymes (proteases, nucleases, etc.) that can attack and destroy proteins or other molecules to be isolated. Special precautions should be taken if working with proteins that are sensitive to degradation or few in number. A common solution to this problem is to include in the solutions protease inhibitors which are physiological (trypsin inhibitors, antipain, leupeptin, etc.) or artificial (E64, PMSF, etc.). Then, various techniques exist to isolate the desired protein.

One of the methods most suited to large volumes is differential precipitation with ammonium sulfate. Ion-exchange or affinity chromatography procedures, also applicable to large sample volumes but having a fairly good separation power, are good intermediate methods. To finalize the purification, molecular sieving or isoelectric focusing is often used. These techniques allow purity to be refined but require very small volumes of concentrated protein. It is often advantageous, between these steps, to remove the salts or products used in these chromatography methods. This can be achieved by dialysis or ultrafiltration.

It may also be advantageous to use a vector with a sequence to identify the PlGF of the invention. In addition, it may be advantageous to facilitate secretion in a prokaryotic or eukaryotic system. Indeed, in this case, the recombinant protein of interest will be present in the cell culture supernatant rather than inside the host cells.

PlGF can be produced at rates of at least 1 mg per liter, preferably 2 mg per liter, even more preferably 5mg per liter of cell culture.

Alternatively, it is possible to prepare PlGF by chemical synthesis. The skilled person is familiar with chemical synthesis processes, for example techniques using solid phases or partial solid phases, by protein condensation or by conventional solution synthesis. The fragments of the invention can, for example, be synthesized by synthetic chemistry techniques, such as Merrifield synthesis, which is advantageous for reasons of purity, antigenic specificity, absence of unwanted by-products and ease of production. This chemical synthesis can be coupled with a genetic engineering approach or by genetic engineering alone using techniques well-known to the skilled person and described for example in Sambrook J. et al., Molecular Cloning: A Laboratory Manual, 1989. Reagents and starting materials are commercially available or can be synthesized by well-known conventional techniques (see for example, WO 00/12508, WO 2005/085260).

In particular, PlGF analogues can be synthesized using the technique described by Zheng et al. (Acta Ophthalmologica 2012 90(7):e512-e523).

As previously indicated, the object of the present invention is a PlGF for use in the prevention and treatment of FASD. The term “fetal alcohol spectrum disorders (FASD)” refers to all disorders in children resulting from alcohol exposure during pregnancy. This term includes, among other things, all behavioral disorders that will gradually appear with age. Children with these disorders are referred to as “FASD children”. In their most severe form, FASD correspond to fetal alcohol syndrome (FAS). The latter results in craniofacial dysmorphism (including shortened palpebral fissures, a smooth, elongated, flattened nasolabial fold and a thin upper lip); nonspecific growth retardation (height or weight or head circumference), which may be prenatal or postnatal or both; variously associated malformations (heart disease, urogenital malformations, digestive malformations or disorders of the brain architecture) and neurological developmental disorders sometimes expressed by mental retardation and more often by learning difficulties. Children with FAS are called “FAS children”.

Most FASD children have maladaptive and behavioral disorders that appear gradually with age (including breastfeeding and eating difficulties, sleep disorders and, at school age, behavioral anomalies, cognitive disorders affecting learning, intellectual deficits with an IQ lowered by 20 points (1 to 2 standard deviations), learning difficulties in reading (dyslexia) and even more in arithmetic (dyscalculia), attention disorders with hyperactivity or hyperkinesia. They are known as attention-deficit/hyperactivity disorder (ADHD). The French term for FASD is “les troubles causés par l'alcoolisation fetale (TCAF)”. A complete definition of FASD is provided in the Report of the French National Academy of Medicine on Fetal Alcohol Exposure (adopted on 22 Mar. 2016).

The inventors have previously shown that alcohol exposure causes cerebrovascular damage. The term “cerebrovascular damage” refers here to any alteration of the cerebral vasculature, in particular an alteration resulting in impaired or even defective functioning of said system. Cerebrovascular damage in the context of the invention may include disorganization of the cerebral vasculature. More particularly, fetal alcohol exposure induces random orientation of the brain vessels. According to a particular embodiment, the fetal alcohol spectrum disorder is associated with cerebrovascular disease. More particularly, said fetal alcohol spectrum disorder is associated with disorganization of the cerebral vasculature.

The inventors have also demonstrated through their previous work that fetal alcohol exposure causes a failure in brain angiogenesis. Brain angiogenesis is the process of blood vessel formation in the brain.

According to one embodiment of the present invention, PlGF is used to stimulate brain angiogenesis and thus improve brain function.

According to still another embodiment of the invention, PlGF can be used to prevent and/or treat at least one FASD selected from any of the above-mentioned maladaptive and behavioral disorders. PlGF can also be used to prevent and/or treat fetal alcohol syndrome (FAS), particularly when FAS manifests as hypotrophy.

In the context of the present invention, the term “fetal alcohol syndrome (FAS)” refers to the most extreme and disabling manifestation of fetal alcohol spectrum disorder (FASD). It combines physical abnormalities such as hypotrophy (growth retardation), craniofacial dysmorphism and neurobehavioral abnormalities resulting in cognitive function disorders (attention, motor, learning or memory disorders). Partial FAS is the case in which the child has only some FAS symptoms. However, partial FAS children still have one or more neurobehavioral abnormalities.

In the context of the invention, an FAS child, particularly a newborn, is considered “hypotrophic” if his or her weight and height are below the 10th percentile of the reference curves. This is often the case for preterm newborns and newborns exposed to alcohol in utero.

Hypotrophy can also be diagnosed before birth by ultrasound: this is called fetal hypotrophy or intrauterine growth retardation.

The inventors have also demonstrated that increasing the amount of PlGF increases the size of the whole body of a fetus or of one or more of its parts, particularly skull size, body size, abdomen size and depth. This allows the partial or complete restoration of the normal morphometric appearance. The administration of PlGF therefore compensates for the hypotrophy of FAS subjects who represent the most severe forms of FASD.

According to another embodiment, the present invention thus concerns a PlGF for use in the prevention and/or treatment of FAS, in particular when FAS manifests as hypotrophy due to intrauterine alcohol exposure. In particular, it is hypotrophy of the whole body of a subject or of one or more of its parts selected from the torso (also called the body), the abdomen and the skull. In particular, the subject is a fetus or a child, especially a preterm one.

According to one embodiment of the present invention, the subject who has been exposed to alcohol in utero is selected from an embryo, a fetus or a child, preferably a fetus or a child, in particular a preterm child.

The period between the 30th week of gestation and the term of pregnancy is the period during which brain angiogenesis is greatest. It is therefore particularly preferred to administer PlGF to a fetus exposed to alcohol in utero at precisely this time.

According to a particular embodiment of the invention, when the FASD subject is a preterm child, treatment will consist in supplementing this subject with PlGF over the period ex utero corresponding to the weeks of intrauterine life lost. Preferably, the treatment window can extend from the 30th week of gestation to the theoretical term (40 weeks' gestation), during which time brain angiogenesis in humans is particularly intense.

According to the invention, the term “subject” means a human being, and preferably an embryo, a fetus or a child. The term “embryo”, as used here, is a fertilized oocyte less than three months old. The term “fetus” refers here to an individual taken before birth and whose gestational age is comprised between 3 and 9 months. After delivery, the subject becomes a child. According to the invention, the term “child” means an individual whose age is less than 3 years. Thus, the category of children according to invention comprises newborns, whose age is between 0 and 1 month, infants, who are between 1 month and 2 years old, and children themselves, who are at least 2 years old. A “newborn”, as used here, can be born at term or be preterm. In the context of the present application, the term “preterm” refers to a child born alive before 37 weeks of amenorrhea. This term covers three subcategories: extremely preterm (<28 weeks); very preterm (28 to 32 weeks); and moderate to late preterm (32 to 37 weeks). In the present invention, preterm children treated with PlGF are preferably in the moderate to late preterm category.

The term “a subject with fetal alcohol spectrum disorder” or “FASD subject” as used here refers to an embryo, a fetus or a subject, particularly a human subject, that is exposed or likely to be exposed to alcohol in utero and that suffers from fetal alcohol spectrum disorder or is at risk of developing, due to maternal alcohol consumption, one of the conditions associated with fetal alcohol spectrum disorder, including the effects described above. In particular, an FASD subject may have a smaller than normal whole body or parts thereof and a disorganized cerebral vasculature, said disorganization being related in particular to a random orientation of the brain vessels. When an FASD subject is affected by FAS in particular, this is referred to as an “FAS subject”.

The term “treatment”, as used here, refers to any action to reduce or eliminate the symptoms or causes of FASD. A treatment in the sense of the invention may include the administration of PlGF, a pharmaceutical composition or a product containing same with or without psychotherapeutic follow-up.

The term “prevention”, as used here, refers to any action that completely or partially prevents the risk of symptoms or causes of FASD. Prevention in the sense of the present invention comprises the administration of PlGF, a pharmaceutical composition, to a subject exposed to alcohol in utero or to a subject who has been exposed to alcohol in utero but for whom symptoms of FASD have not yet appeared and brain angiogenesis is still ongoing. The inventors take advantage of the fact that PlGF is a naturally occurring angiogenic factor at these stages of fetal development in a healthy subject, which has the advantage of easy use of PlGF by compensation in the prevention of FASD.

According to one embodiment of the present invention, placental growth factor is administered in a therapeutically effective amount to a subject who has been exposed to alcohol in utero.

In the context of the invention, “a therapeutically effective amount” means an amount sufficient to influence the therapeutic course of a particular disease condition. A therapeutically effective amount is also the amount at which all toxic or secondary effects of the agent are offset by the therapeutically beneficial effects of the active principle used.

According to another embodiment of the present invention, placental growth factor (PlGF) is administered in utero and/or ex utero. It is therefore possible to start treatment (or prevention) by administering PlGF during the intrauterine period and to continue it after delivery, especially when the child is born prematurely and consequently loses the physiological supply of placental PlGF.

Preferably, PlGF will be administered alone or in a pharmaceutical composition by the systemic route, in particular by the intravenous, intramuscular, intradermal, intraperitoneal, subcutaneous or oral route. More preferably, PlGF will be administered several times, spread over time.

In particular, when PlGF is administered in utero, it is preferably administered directly to the placenta in order to reach the fetal brain more quickly and to avoid degradation of the protein by the maternal organism.

When PlGF is administered after birth, it is preferably administered parenterally, preferably intravenously, especially in the case of a preterm child or newborn.

Optimal modes of administration, dosages and dosage forms can be determined according to the criteria generally taken into account in the establishment of a treatment adapted to a patient, such as the patient's age or body weight, the severity of his or her general condition, tolerance to the treatment and the side effects observed.

When PlGF is administered as pharmaceutical compositions, for subcutaneous, intramuscular, intramuscular, intravenous, local transdermal administration, PlGF may be administered as unit dosage forms, mixed with conventional pharmaceutical carriers to the subject in need thereof. Appropriate unit forms of administration include intramuscular, intravenous forms.

As indicated above, the choice of the most appropriate route of administration will depend on when this administration is carried out. In particular, when the administration of a cosmetic composition containing PlGF is carried out in utero, it will be administered by the intermuscular or intravenous route, preferably to the placenta. The administration of the pharmaceutical composition containing PlGF to a newborn or a preterm child is preferably done intravenously.

For parenteral, intranasal or intraocular administration, aqueous suspensions, isotonic saline solutions or sterile injectable solutions containing pharmacologically compatible dispersing agents and/or wetting agents are used.

The forms for parenteral administration are obtained conventionally by mixing PlGF with buffers, stabilizers, preservatives, solubilizers, isotonic agents and suspending agents. In accordance with known techniques, these mixtures are then sterilized and packaged as intravenous injections.

As buffer, the skilled person may use buffers based on organic phosphate salts. Examples of suspending agents include methylcellulose, hydroxyethylcellulose, hydroxypropylcellulose, acacia and sodium carboxymethylcellulose. In addition, stabilizers useful according to the invention are sodium sulfite and sodium metabisulfite, while sodium p-hydroxybenzoate, sorbic acid, cresol and chlorocresol are examples of preservatives.

The pharmaceutical compositions of the invention may be formulated to be administered to the patient by a single route or by different routes.

The dosage naturally depends on the form in which PlGF will be administered, the method of administration, the therapeutic indication, the patient's age and condition.

The dose to be administered is preferably from 0.001 to 250 mg/kg PlGF per day, preferably from 0.01 to 100 mg/kg PlGF per day, more preferably from 0.1 to 50 mg/kg PlGF per day and even more preferably 1 to 25 mg/kg PlGF per day.

The unit dose of PlGF preferably includes 0.1 to 50 mg/kg of this compound.

According to one embodiment of the present invention, the initial dose of PlGF administered may if necessary be adjusted during treatment according to the response of the treated subject to this treatment. Based on this general knowledge and on the present description, the skilled person will be able to adjust the dose of PlGF so as to optimize its therapeutic effect.

As mentioned above in the description, placental growth factor (PlGF) can also be used as active principle in a pharmaceutical composition.

According to a second aspect, the present invention thus concerns a pharmaceutical composition comprising a placental growth factor (PlGF) as defined above and a pharmaceutically acceptable carrier for use in the prevention and/or treatment of fetal alcohol spectrum disorders (FASD).

The pharmaceutical composition according to the present invention may be used in the prevention and/or treatment of different types of FASD as described for PlGF below. In particular, these FASD are selected from the group consisting of maladaptive and behavioral disorders or fetal alcohol syndrome (FAS), particularly when it manifests as hypotrophy and cerebrovascular damage due to in utero alcohol exposure.

The composition of the present invention may also be used to improve the brain angiogenesis of a subject who has been exposed to alcohol in utero.

In the sense of the present invention, “pharmaceutically acceptable carrier” means any material that is suitable for use in a pharmaceutical product. Examples of pharmaceutically acceptable carriers include lactose, optionally modified starch, cellulose, hydroxypropylmethyl cellulose, mannitol, sorbitol, xylitol, dextrose, calcium sulfate, calcium phosphate, calcium lactate, dextrates, inositol, calcium carbonate, glycine, bentonite and mixtures thereof.

The pharmaceutical composition according to the invention may take different forms and be administered in different ways as indicated in detail above.

According to one embodiment, the pharmaceutical composition of the present invention comprises a PlGF as active principle in a concentration comprised between 0.001 mg/kg and 250 mg/kg by weight based on the total weight of the subject to whom the pharmaceutical composition will be administered.

Preferentially, the PlGF concentration is comprised between 0.01 mg/kg and 100 mg/kg by weight based on the weight of the subject to whom the pharmaceutical composition will be administered and, more particularly, between 0.15 mg/kg and 50 mg/kg by weight based on the weight of the subject to whom the pharmaceutical composition will be administered or between 1 mg/kg and 25 mg/kg by weight based on the total weight of the subject to whom the composition will be administered.

The inventors have demonstrated that increasing the amount of PlGF is particularly effective in restoring brain vascularization damage, thus restoring neuronal function to a normal state.

According to one embodiment of the present invention, PlGF or the pharmaceutical composition comprising same is therefore used for the treatment and/or prevention of brain vascularization damage due to in utero alcohol exposure.

In addition, the inventors have also demonstrated that increasing the amount of PlGF increases the size of the whole body of a fetus or of one or more of its parts, in particular skull size, body size, abdomen size and depth after alcohol exposure during the intrauterine period. This allows the partial or complete restoration of the normal morphometric appearance.

According to another embodiment, the present invention thus concerns a PlGF or a pharmaceutical composition comprising same for use in the prevention and/or treatment of hypotrophy due to intrauterine alcohol exposure, in particular when this hypertrophy is a manifestation of FAS. In particular, it is a hypotrophy of the whole body of a subject or of one or more of its parts selected from the torso (also called the body), the abdomen and the skull. In particular, the subject is an FAS subject who is a fetus or newborn, particularly a preterm one.

A fourth aspect of the invention concerns a method for treating fetal alcohol spectrum disorders in a subject. This method comprises a step of administering or overexpressing PlGF or administering a pharmaceutical composition comprising same to a subject with fetal alcohol syndrome (FAS), in particular when it manifests as hypotrophy and/or cerebrovascular damage in a subject who has been exposed to alcohol in utero.

This treatment method may include a preliminary step of diagnosing FASD.

According to a fifth aspect, the present invention concerns placental growth factor (PlGF), a pharmaceutical composition or a product comprising same according to the invention for use in the treatment of FASD, said use including a step prior to the identification of the subject, said identification comprising the following steps:
a) measurement of the amount of PlGF in a biological sample from said subject, preferably from the placenta or umbilical cord blood;
b) comparison of the amount of PlGF in step a) with a reference that is a measure of the amount of PlGF in a healthy individual, and
c) determination of an FASD or of the risk of developing an FASD in said subject.

According to the invention, “biological sample” means any sample that can be collected from a subject. Alternatively, the biological sample is a sample from the placenta, particularly from the umbilical cord. Indeed, PlGF is expressed by placental cells throughout pregnancy. This allows PlGF to be measured without compromising the subject's integrity, especially when the subject is an embryo or fetus. In general, the biological sample must allow the determination of the PlGF expression rate. The test sample may be used as obtained directly from the biological source or following pretreatment to modify the character of the sample. For example, such pretreatment may include the preparation of plasma from blood, dilution of viscous fluids and so on. Pretreatment processes may also involve filtration, precipitation, dilution, distillation, mixing, concentration, inactivation of disruptive components, addition of reagents, lysis, etc. In addition, it may be beneficial to modify a solid test sample to form a liquid medium or to release the analyte.

The PlGF protein is a secreted protein (DeFalco, Exp Mol Med. 44(1):1-9, 2012). The preferred biological samples for the determination of the expression rate of said protein include in particular blood, plasma, or lymph samples. Preferably, the biological sample is a blood sample. Even more preferably, the biological sample is a sample of placental blood or of umbilical cord blood. The latter is usually collected during delivery. Blood from the placental vessels can then be obtained to measure the PlGF level.

According to one embodiment, when the amount of PlGF measured in step a) is less than the reference, the subject is determined to have or to be at risk of developing FASD and in particular brain vascularization damage or FAS, particularly hypotrophy.

According to the present invention, the amount of PlGF is measured when a subject has at least one disorder that may be associated with intrauterine alcohol exposure. The amount of PlGF will also be measured when a subject does not have one or more specific disorders associated with intrauterine alcohol exposure but for whom alcohol exposure during this period has been proven or assumed. Thus, measuring the amount of PlGF as described above will confirm or rule out a FASD or the risk of developing one before therapeutic management.

According to one embodiment, the amount of PlGF is determined by measuring the number of transcripts encoding PlGF or the amount of the polypeptide.

The level of gene or protein expression can be measured by many methods that are available to the skilled person. There may be several intermediate steps between collecting the biological sample and measuring PlGF expression, said steps corresponding to the extraction from said sample of an mRNA (or corresponding cDNA) sample or a protein sample. This can then be used directly to measure PlGF expression. The preparation or extraction of mRNA (as well as its reverse transcription into cDNA) or proteins from a cell sample are but routine procedures well-known to the skilled person.

Once an mRNA (or corresponding cDNA) or protein sample is obtained, PlGF expression at the level of either mRNA (i.e. all mRNAs or cDNAs present in the sample) or proteins (i.e. all proteins present in the sample) can be measured. The method used to that end thus depends on the type of transformation (mRNA, cDNA or protein) and the type of sample available.

When PlGF expression is measured at the mRNA (or corresponding cDNA) level, any technology usually used by the skilled person can be employed. These technologies for analyzing the level of gene expression, such as transcriptome analysis, include well-known methods such as the polymerase chain reaction (PCR, if starting from DNA), reverse transcription-PCR (RT-PCR, if starting from RNA) or quantitative RT-PCR or nucleic acid chips (including DNA chips and oligonucleotide chips) for higher throughput.

The term “nucleic acid chips” refers here to several different nucleic acid probes that are attached to a substrate, which may be a microchip, a glass slide, or a microsphere-sized bead. The microchip can be made of polymers, plastics, resins, polysaccharides, silica or a material based on silica, carbon, metals, inorganic glass, or nitrocellulose.

Probes can be nucleic acids such as cDNA (“cDNA chip”), mRNA (“mRNA chip”) or oligonucleotides (“oligonucleotide chip”), which oligonucleotides may typically have a length comprised between about 25 and 60 nucleotides.

To determine the expression profile of a particular gene, a nucleic acid corresponding to all or part of said gene is labelled and then brought into contact with the chip under hybridization conditions, leading to the formation of complexes between said labelled target nucleic acid and probes attached to the chip surface that are complementary to that nucleic acid. The presence of labelled hybrid complexes is then detected.

These technologies make it possible to monitor the level of expression of a particular gene or several genes or even all the genes in the genome (full genome or full transcriptome) in a biological sample (cells, tissues, etc.). These technologies are routinely used by the skilled person and therefore there is no need to detail them here.

Alternatively, it is possible to use any current or future technology allowing gene expression to be determined based on the amount of mRNA in the sample. For example, the skilled person can measure the expression of a gene by hybridization with a labelled nucleic acid probe, such as northern blot (for mRNA) or Southern blot (for cDNA), but also by techniques such as the serial analysis of gene expression (SAGE) method and derivatives thereof, such as LongSAGE, SuperSAGE, DeepSAGE, etc. It is also possible to use tissue chips (also known as tissue microarrays, TMAs). The tests usually used with tissue chips include immunohistochemistry and fluorescence in situ hybridization. For mRNA analysis, tissue chips can be coupled with fluorescence in situ hybridization. Finally, it is possible to use massive parallel sequencing to determine the amount of mRNA in the sample (RNA-Seq or whole-transcriptome shotgun sequencing). To that end, several massive parallel sequencing methods are available. Such methods are described in, for example, U.S. Pat. Nos. 4,882,127, 4,849,077; 7,556,922; 6,723,513; WO 03/066896; WO 2007/111924; US 2008/0020392; WO 2006/084132; US 2009/0186349; US 2009/0181860; US 2009/0181385; US 2006/0275782; EP-B1-1141399; Shendure Et Ji, Nat Biotechnol., 26(10):1135-45. 2008; Pihlak et al., Nat Biotechnol., 26(6): 676-684, 2008; Fuller et al., Nature Biotechnol., 27(11):1013-1023, 2009; Mardis, Genome Med., 1(4):40, 2009; Metzker, Nature Rev. Genet., 11(1):31-46, 2010.

Preferably, PlGF expression is measured at the protein level by a method selected from immunohistology, immunoprecipitation, western blot, dot blot, ELISA or ELISPOT, protein chips, antibody chips, or tissue chips coupled with immunohistochemistry, FRET or BRET techniques, microscopy or histochemistry methods, including in particular confocal and electron microscopy methods, methods based on the use of one or more excitation wavelengths and an appropriate optical method, such as an electrochemical method (voltammetric and amperometric techniques), atomic force microscopy, and radiofrequency methods, such as multipolar, confocal and non-confocal resonance spectroscopy, detection of fluorescence, luminescence, chemiluminescence, absorbance, reflectance, transmittance, and birefringence or refractive index (for example, by surface plasmon resonance, by ellipsometry, resonant mirror method, etc.), flow cytometry, radioisotopic or magnetic resonance imaging, polyacrylamide gel electrophoresis analysis (SDS-PAGE); by HPLC-mass spectrophotometry, by liquid chromatography/mass spectrophotometry/mass spectrometry (LC-MS/MS).

More preferentially, the amount of PlGF is determined by a method selected from immunoprecipitation, immunohistology, western blot, dot blot, ELISA or ELISPOT, protein chips, antibody chips, or tissue chips coupled with immunohistochemistry. Antibodies directed against PlGF are commercially available (see, for example, R&D Systems, Santa Cruz, Abcam, etc.) and can be used in the methods of the invention. Even more preferably, PlGF expression is measured by western blot or ELISA.

In addition, the amount of PlGF is normalized to a control marker which may be a gene selected from B2M, TFRC, YWHAZ, RPLO, 185, GUSB, UBC, TBP, GAPDH, PPIA, POLR2A, ACTB, PGK1, HPRT1, IPO8 and HMBS, or a polypeptide selected from the product of said genes.

The measured PlGF rate is then compared with a reference PlGF expression level to determine if it is an FASD subject.

In the sense of the present application, “a reference PlGF expression rate” means any expression rate of said factor used as a reference. For example, a reference expression rate can be obtained by measuring the expression rate of PlGF in a biological sample, for example a placenta or umbilical cord blood, from a healthy subject, meaning a subject who has not been exposed to alcohol in utero.

The invention will be described more precisely using the examples below. Said examples are provided here by way of illustration only and are not, unless otherwise indicated, intended to be limiting.

### FIGURE LEGENDS

FIG. 1: Effects of in utero alcohol exposure on cortical angiogenesis in E20 mouse embryos. A, B: Effects of fetal alcohol exposure from GD15 to GD20 on cortical microvessel organization in control (A) and alcohol-exposed animals (B). Brain microvessels were visualized by anti-CD31 immunohistochemistry. The arrows indicate brain microvessels with radial orientation in the “Control” group. It should be noted that there is a loss of radial organization in the “Alcohol” group. I-VI: Cortical layers; CC: Corpus callosum. C: Distribution of the orientation (angle classes) of cortical microvessels in the immature cortex of GD20 fetuses. Statistical analysis was performed using the x² test. D: Western blot quantification of the effects of fetal alcohol exposure during the last week of gestation on cortical expression of CD31 at GD20. ns vs. “Control” group using an unpaired t-test.

FIG. 2: Effects of in utero alcohol exposure on the expression of VEGF/PlGF family members in E20 mouse embryos. A-E: Western blot quantification of VEGFA (A), PlGF (B), sVEGF-R1 (C), mVEGF-R1 (D) and VEGF-R2 protein levels in the cortex of the “Control” and “Alcohol” groups. F: Western blot comparison of PlGF protein levels in the cortex and placenta of E20 embryos of the “Control” group. *p<0.05; ***p<0.001 vs. “Control” group using an unpaired t-test.

FIG. 3: Effects of in utero alcohol exposure on the ultrastructural features of the placenta in GD20 mice. A: Observation by cresyl violet staining of the effect of alcohol exposure on the laminar structure of the placenta. The maternal side of the placenta is facing upwards. Alcohol affects the segregation of junctional and labyrinth zones (dotted lines). B: Image analysis quantification of the effects of alcohol on Reichert's membrane thickness. C, D: Low magnification observation of the giant trophoblast layer in the “Control” (C) and “Alcohol” (D) groups. The arrows indicate giant trophoblasts. The latter have a typical rectangular shape in the placenta of the “Control” group, while in the “Alcohol” group they have a rounded shape. E-H: Images acquired by electron microscopy at medium (E, F) and high magnification (G, H) showing the cell morphology of giant trophoblasts and the presence of tight junctions (arrows) in the “Control” (E, G) and “Alcohol” (F, H) groups. Tight junctions (stars) is no longer visible in alcohol-treated animals. The insets in E and F indicate the zone observed at higher magnification in G and H, respectively. D: maternal decidua; J: junctional zone; L: labyrinth zone; Tg: giant trophoblast layer. ***p<0.001 vs. “Control” group using an unpaired t-test.

FIG. 4: Effects of in utero alcohol exposure on the expression of proteins involved in the placental barrier and in placental energy metabolism. A, B: Immunohistochemical observation of ZO-1 protein in the placental labyrinth zone of mice in the “Control” (A) and “Alcohol” (B) groups. ZO-1 protein appears as forming groups of dots (arrows) in the “Control” group while the staining is diffuse in the “Alcohol” group. The trophoblast layers were revealed by immunoreactivity with the glucose transporter Glut-1. Nuclei were stained with Hoechst. C: Double staining with antibodies against monocarboxylate MCT-1 and glucose transporters in the labyrinth zone of a “Control” placenta. In contrast to Glut-1, MCT-1 expression is associated with the maternal layer of the syncytiotrophoblast. Nuclei were stained with Hoechst. D: Western blot quantification of ZO-1 and MCT-1 protein expression levels in the placentas of the “Control” and “Alcohol” groups. *p<0.05, “p<0.01 vs. control group using an unpaired t-test. Western blot analyses showed that the ZO-1 level decreased significantly in placentas of alcohol-exposed animals while the MCT-1 protein level increased significantly. *p<0.05, “p<0.01 compared with the control group using the unpaired t-test. E-H: Immunohistochemistry tests illustrate the distribution of VEGF-R1 (E), Glut-1 (F, G) and PlGF (H) in the syncytiotrophoblast layers of the mouse placenta. Nuclei were stained with Hoechst.

FIG. 5: Effects of in utero alcohol exposure on the expression of VEGF/PlGF family members in murine placentas. A-F: Western blot quantification of the effects of alcohol exposure during the last week of gestation on placental expression of VEGF-A (A), PlGF (B), sVEGF-R1 (C), mVEGF-R1 (D), VEGF-R2 (E) and CD31 (F) at GD20. G, H: Immunohistochemical staining showing the distribution of VEGF-R2 (G) in the syncytiotrophoblast layers of the placenta stained with Glut-1 (H). Nuclei were stained with Hoechst. *p<0.05 vs. “Control” group using an unpaired t-test.

FIG. 6. Diffusion of Evans blue and recombinant human PlGF injected in utero from the placenta to the fetal brain and effect of placental suppression of PlGF on brain vascularization. A, B: Time-course visualization of Evans blue administered by microinjection into the placenta of pregnant mice at GD15. Fluorescence was detected by UV illumination (A) and is represented using a false-color scale (B). C, D: Time-course visualization of Evans blue fluorescence in the fetal brain after placental microinjection at GD15. Fluorescence was detected by UV illumination (C) and is represented using a false-color scale (D). E, F: Time-course spectrophotometric quantification of the absorbance at 595 nm of the signal of the Evans blue injected into the placentas (E) and then into the brains of the corresponding fetuses (F). G: ELISA quantification of human PlGF in fetal mouse brain 30 min after injection of hPlGF into the placentas of pregnant mice at GD15. *p<0.05 vs. “Control” group using an unpaired t-test. H: Microphotography visualizing eGFP expression 48 hours after in utero transfection of GD15 pregnant mouse placentas with a plasmid encoding an eGFP. I, J: Triple staining with eGFP/Glut-1/Hoechst indicating that eGFP fluorescence (I) is mainly associated with the fetal trophoblast layer (J) stained with Glut-1 (arrowheads). The fetal part of the trophoblast layers is identified by the presence of specific nucleated red blood cells of the fetal circulation (arrows). K: Western blot visualization of PlGF, GFP and actin proteins in the placentas of untransfected (sh−/GFP−), GFP-transfected (sh−/GFP+) and shPLGF/GFP-transfected (sh+/GFP+) animals. L, M: Western Blot quantification of PlGF levels (L) and GFP expression (M) in placentas of untransfected (sh−/GFP−), GFP-transfected (sh−/GFP+) and shPLGF/GFP-transfected (sh+/GFP+) animals four days after transfection. N: Western transfer quantification of VEGF-R1 expression levels in the fetal brain from untransfected (sh−/GFP−), GFP-transfected (sh−/GFP+) and shPLGF/GFP-transfected (sh+/GFP+) placentas four days post-transfection. *p<0.05 vs. “sh−/GFP” group using one-way ANOVA followed by Tukey's post-hoc test. O-R: Visualization of the vasculature in the fetal cortex from untransfected (Sh−/GFP−) (O), GFP-transfected (Sh−/GFP+) (P) and shPLGF/GFP-transfected (Sh+/GFP+) placentas. (Q) Statistical analysis of cortical vessel disorganization performed using the x² test (R).

FIG. 7. Morphometric characterization of the effects of in utero alcohol exposure on gestational week 20 to 25 human placenta. A, B: Anti-CD31 immunohistochemical staining and toluidine blue counterstaining to visualize the microvessels (brown) present in the placental villi (blue) of the “Control” (A) and “FAS/pFAS” (B) groups at gestational ages [20-25 WG[. C: Percentage of villi classified by size in the placentas of the “Control” and “FAS/pFAS” groups at gestational ages [20-25 WG[. D: Vessel distribution by villus size in the placentas of the “Control” and “FAS/pFAS” groups at gestational ages [20-25 WG[. E: Vascular surface area by villus size in the placentas of the “Control” and “FAS/pFAS” groups at gestational ages [20-25 WG[. *p<0.05 vs. “Control” group using an unpaired t-test.

FIG. 8. Morphometric characterization of the effects of in utero alcohol exposure on gestational week 25 to 35 human placenta. A, B: Anti-CD31 immunohistochemical staining and toluidine blue staining to visualize the microvessels (brown) present in the placental villi (blue) of the “Control” (A) and “FAS/pFAS” (B) groups at gestational ages [25-35 WG[. C: Percentage of villi classified by size in the placentas of the “Control” and “FAS/pFAS” groups at gestational ages [25-35 WG[. D: Vessel distribution by villus size in the placentas of the “Control” and “FAS/pFAS” groups at gestational ages [25-35 WG[. E: Vascular surface area by villus size in the placentas of the “Control” and “FAS/pFAS” groups at gestational ages [25-35 WG[. *p<0.05 vs. “Control” group using an unpaired t-test.

FIG. 9. Morphometric characterization of the effects of in utero alcohol exposure on gestational week 35 to 42 human placenta. A, B: Anti-CD31 immunohistochemical staining and toluidine blue staining to visualize the microvessels (brown) present in the placental villi (blue) of the “Control” (A) and “FAS/pFAS” (B) groups at gestational ages ranging from [35-42 WG[. The microvessel lumen area is greatly reduced in the “FAS/pFas” group. C: Percentage of villi classified by size in the placentas of the “Control” and “FAS/pFAS” groups at gestational ages ranging from [35-42 WG[. D: Vessel distribution by villus size in the placentas of the “Control” and “FAS/pFAS” groups at gestational ages ranging from [35-42 WG[. E: Vascular surface area by villus size in the “Control” and “FAS/pFAS” groups at gestational ages ranging from [35-42 WG[. *p<0.05 vs. “Control” group using an unpaired t-test.

FIG. 10: Time-course effects of in utero alcohol exposure on villus and vessel densities in human placentas and western blot characterization of pro-angiogenic proteins and energy metabolism. A: Change in villus densities in the placentas of the “Control” (A) and “FAS/pFAS” (B) groups at gestational ages [20-25 WG[, [25-35 WG[ and [35-42 WG[. B: Change in vessel densities in the “Control” and “FAS/pFAS” groups at gestational ages [20-25 WG[, [25-35 WG[ and [35-42 WG[. ^{#}p<0.05, ^{##}p<0.01 vs. “Control” group as indicated on the graph. *p<0.05, ***p<0.001 for the “Control” vs. “Alcohol” groups for a given gestational age group. C-H: Western blot quantification of ZO-1 (C), MCT-1 (D), PlGF (E), VEGFA (F), VEGF-R1 (G) and VEGF-R2 (H) protein levels in the placentas of the “Control” and “FAS/pFAS” groups. *p<0.05 vs. “Control” group using an unpaired t-test.

FIG. 11. Comparison of cerebral and placental damage observed in human fetuses and induced by in utero alcohol exposure and statistical correlation. A-H: Vascular organization in the brains (A, D) and the placentas (E, H) of patients in the “Control” group at WG22 (A, E) and WG31 (C, G) and vascular organization in brains (B, D) and placentas (F, H) of patients in the “FAS/pFAS” group at WG21 (B, F) and WG33 (D, H). I, J: Statistical correlation between cortical vascular disorganization and placental vascular density in patients in the “Control” (I) and FAS/pFAS (J) groups.

FIG. 12. Effects of in utero PlGF overexpression on fetal growth and cortical vascularization during intrauterine alcohol exposure. A, B: A PGF/CRISPR/dCas9 activation approach coupled with in utero electroporation of the placenta was performed at GD13 (A) and PlGF overexpression was checked at GD20 (B). In the “Alcohol” group, in utero alcohol exposure occurs between GD15 and GD20. C, D: Visualization of E20 fetuses from pregnant mice exposed to NaCl (C) or alcohol (D). The small size of the alcohol-exposed fetuses should be noted. The bars indicate the morphometric measurements made (head size (a); body size (b), abdomen size (c) and size of the whole fetus (a+b)). E, F: Visualization of E20 fetuses from pregnant mice after in utero electroporation of PGF/CRISPR-dCas9 plasmids in the placentas of “Control” groups (E) or of alcohol-exposed pregnant mice (F). G, H: Quantification of the size of the abdomen (G) and the whole fetus (H) in the “Control” (NaCl) group and in the “Alcohol” group. In the same uterine horn some placentas were not electroporated (black bars), others were electroporated with the control CRISPR-cas9 plasmid (gray bars) or electroporated with PGF/CRISPR-dCas9 plasmids (white bars) ## p<0.01; ### p<0.001; #### p<0.0001 vs. “Control” group and *p<0.05; **p<0.01; ****p<0.0001 as indicated using two-way ANOVA followed by Tukey's post-hoc test. I-K: Visualization of the vasculature in the cortex of the E20 fetus from control (NaCl)/untransfected placentas (I), alcohol/control for CRISPR-cas9 transfected (J) and alcohol PGF/CRISPR-dCas9 transfected. It is noted that PlGF overexpression in the placenta corrects the disorganization of brain vascularization induced by in utero alcohol exposure. L: Quantification of the percentage of radial vessels in the cortex of E20 fetuses from placentas not electroporated (black bars), electroporated with CRISPR-cas9 control plasmids (gray bars) and electroporated with PGF CRISPR-dCas9 plasmids (white bars). #p<0.05; ##p<0.01 vs. “Control” group and *p<0.05 as indicated using two-way ANOVA followed by Tukey's post-hoc test.

FIG. 13. Effects of placental PlGF overexpression on head and body size of the E20 fetus in the “Control” and “Alcohol” groups. A, B: Quantification of head (A) and body (B) size in the “Control” (NaCl) and “Alcohol” groups. In the same uterine horn, some placentas were not electroporated (black bars), others were electroporated with CRISPR-cas9 control plasmids (gray bars) or electroporated with PGF CRISPR-dCas9 plasmids (white bars). ## P <0.01; ### P <0.001; #### P <0.0001 vs. “Control” group and * p<0.05; ** p<0.01 as indicated using two-way ANOVA followed by Tukey's post-hoc test.

### EXAMPLES

### Examples A: Abnormalities Following in Utero Alcohol Exposure

Examples A below include several test results from tests performed prior to the present invention showing that in mice and humans:
fetal alcohol exposure impacts brain angiogenesis and organization of the cerebral vasculature,
these brain alterations are correlated with vascular abnormalities of the placenta,
a placental pro-angiogenic factor is able to reach the fetal brain,
neurodevelopmental abnormalities of brain angiogenesis in FASD children are associated with dysregulation of the placental PlGF/brain VEGF-R1 system,
placental invalidation of PlGF reproduces the effects of fetal alcohol exposure on brain VEGF-R1, and
dysregulation of placental PlGF levels following fetal alcohol exposure predicts brain damage.

Abnormalities in Brain Angiogenesis Following in Utero Alcohol Exposure

Effects of in Utero Alcohol Exposure on Development of the Cerebral Vasculature

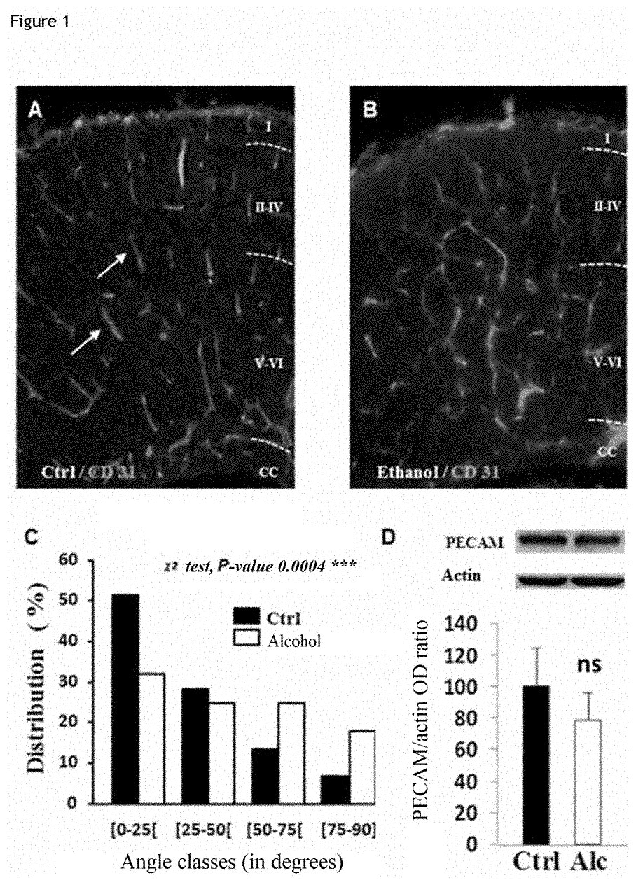

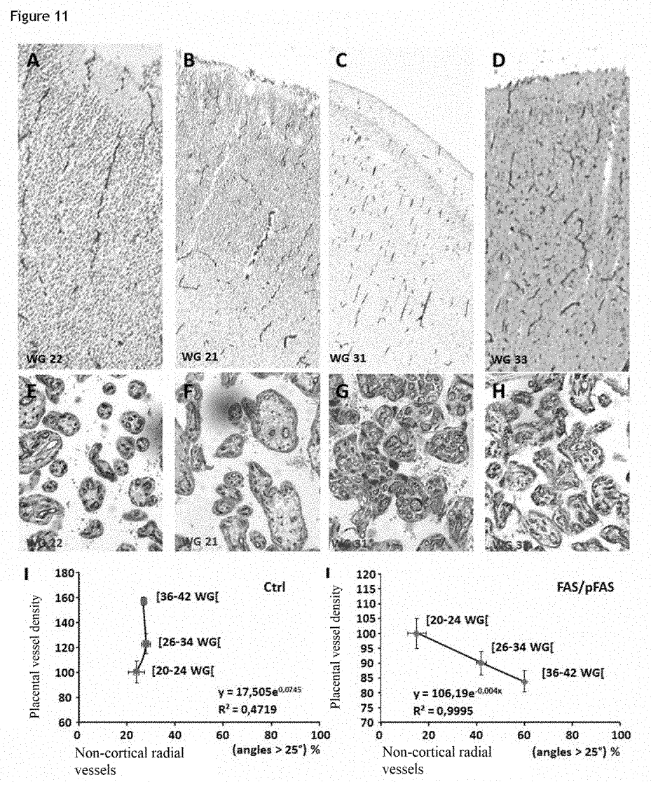

The present inventors have previously demonstrated that prenatal alcohol exposure induces cerebrovascular disorganization. In particular, the effect of alcohol is associated with a significant decrease in the number of cortical vessels with radial orientation and an increase in the number of microvessels with random orientation (FIG. 1). In parallel with the study in mice, an analysis of the cerebral microvasculature in humans showed that, as in mice, cortical microvessels that have a radial orientation in the “Control” group are totally disorganized in the “FAS/pFAS” group (FIG. 11 and Jegou et al., vol. 72, no. 6, 31 Dec. 2012, pages 952-960).

Effects of in Utero Alcohol Exposure on the Expression of Genes Representative of the Vasculature in Mice

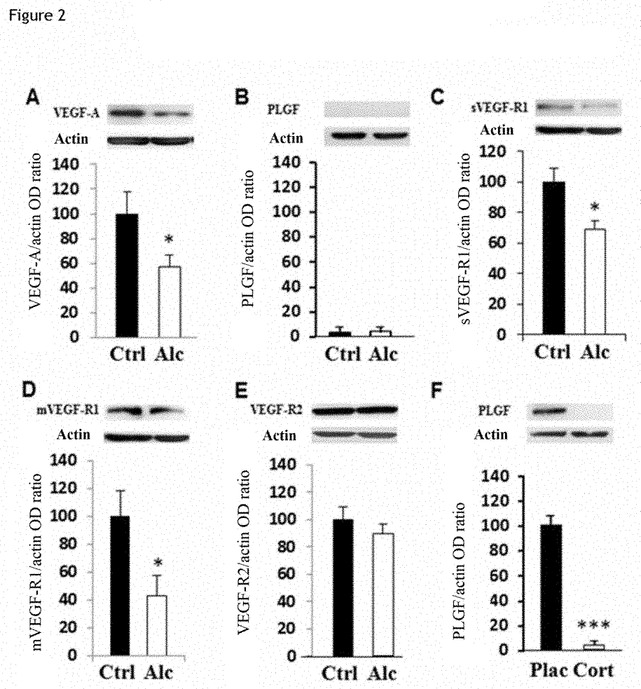

The quantitative RT-PCR (mRNA) and western blot (protein) studies revealed a stained dysregulation of VEGF-R1 and VEGF-R2 receptor levels that relay the pro-angiogenic effects of factors such as VEGFA or PlGF. Cerebral vasculature abnormalities are therefore associated with dysregulation of the expression of pro-angiogenic brain receptors (FIG. 2 and Jegou et al., vol. 72, no. 6, Dec. 31, 2012, pages 952-960).

Abnormalities in Placental Angiogenesis Following in Utero Alcohol Exposure

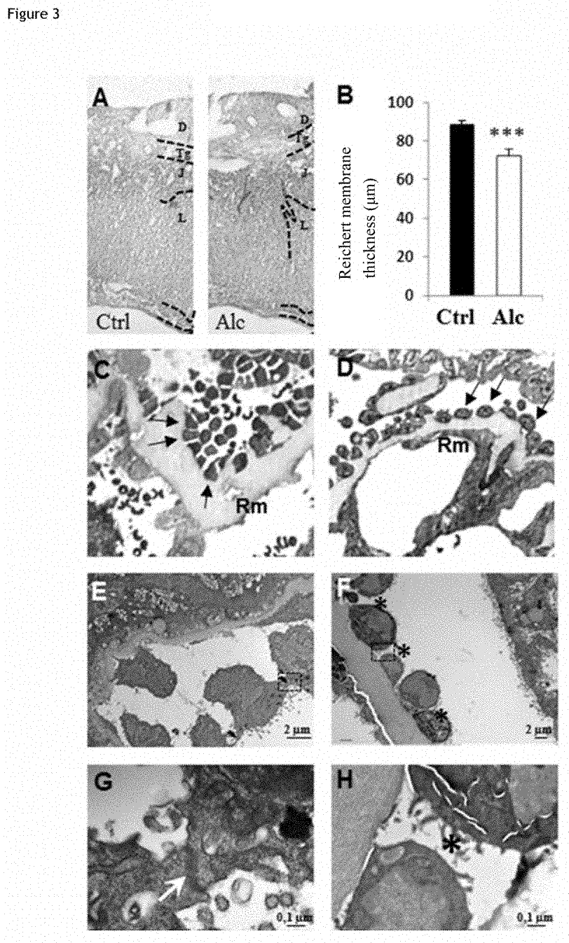

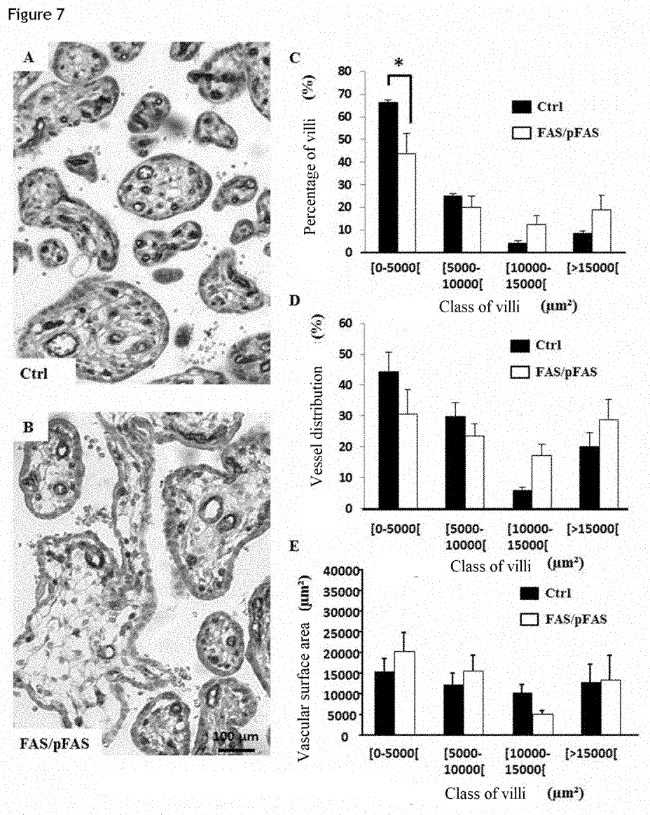

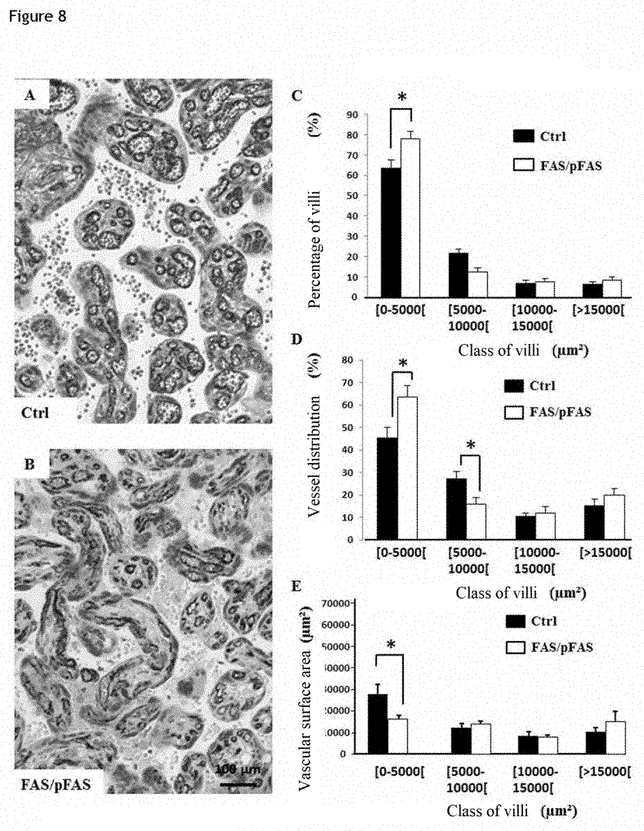

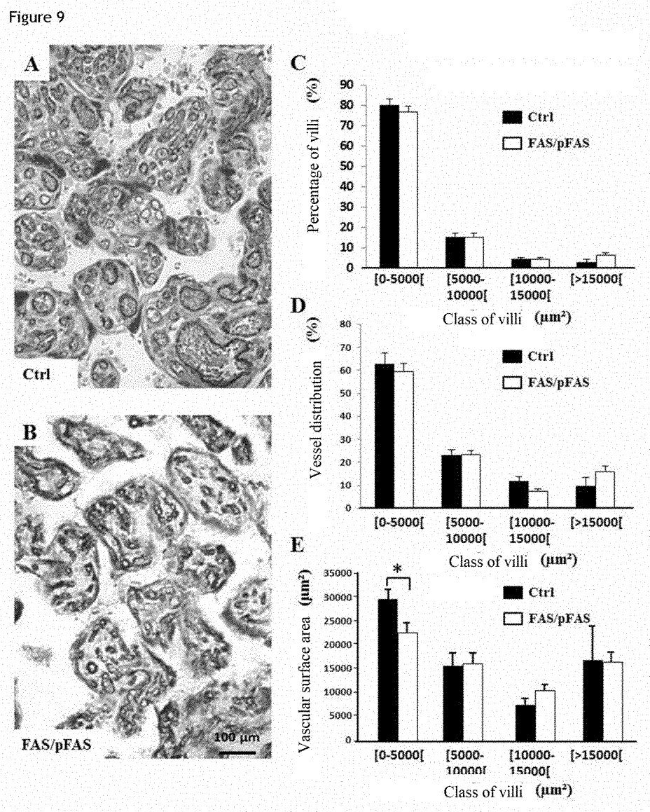

Different placental parameters were studied in mice (FIGS. 3-5) and in humans (FIGS. 7-9) by an immunohistochemical approach coupled with a morphometric analysis notably including placental villus density and size, vascular density and surface area, and the proportion of vessels per villus. In humans, these parameters were measured and compared between 34 placentas of control individuals and 36 placentas from individuals exposed to alcohol in utero.

The placentas were divided into three age groups comparable to those of the brain study (Jegou et al., vol. 72, no. 6, Dec. 31, 2012, pages 952-960). This document presents the results for the age groups [20-25GW[, [25-35GW] and [35-42GW].

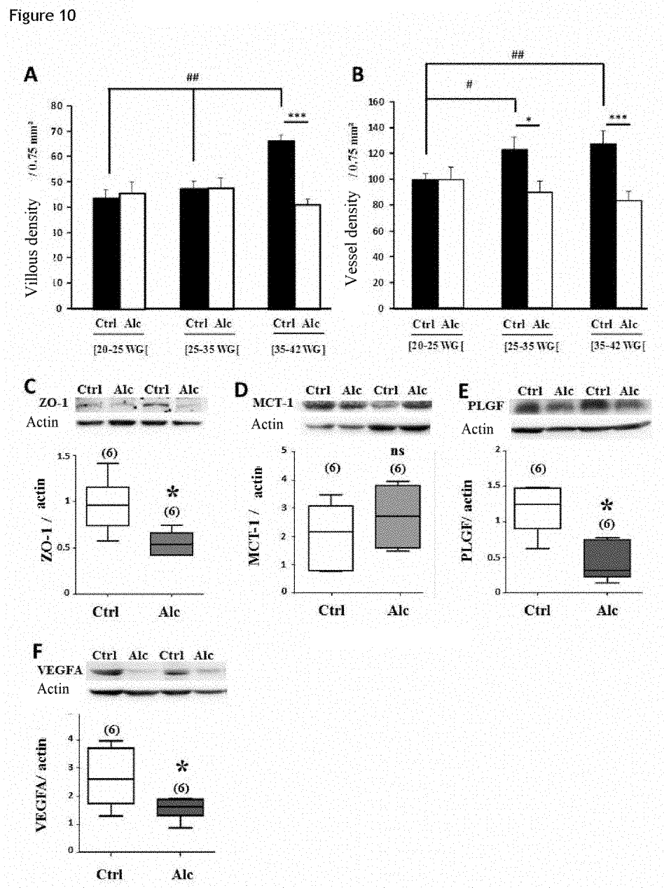

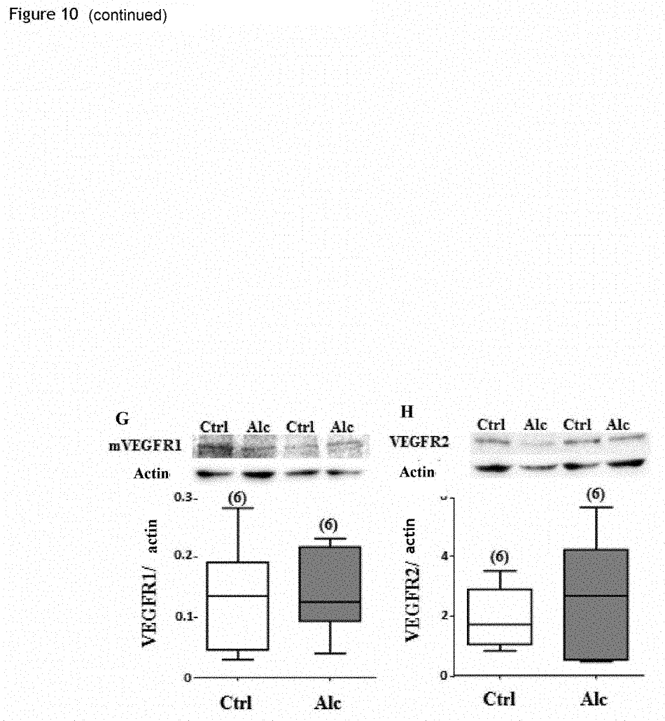

In particular, morphometric analysis indicates that the distribution of placental vessels by villus size and vascular surface area are significantly impacted by alcohol exposure (FIG. 10). In addition, a longitudinal analysis of vascular density taking into account the “age” factor indicates that in the “Control” group placental angiogenesis strongly increases between age groups [20-25GW[ and [25-35GW[. This substantial increase in placental vascularization is due to substantial brain development during the third trimester of pregnancy which requires an increased oxygen and nutrient supply. On the other hand, fetal alcohol exposure induces stagnation or a decrease in placental vascular density (FIG. 10).

In conclusion, the present results indicate that vascular abnormalities exist in both the human placenta and the cerebral cortex in subjects who were exposed to alcohol. These results therefore support the hypothesis of a correlation between brain disorders and placental deficits in angiogenesis.

Demonstration of a Correlation Between Placental and Cerebral Vascular Abnormalities

Placental and cerebral vascular abnormalities observed in humans following in utero alcohol exposure may be the result of totally independent processes with no causal relationship or, conversely, may be closely related. The fact that the PlGF source is unique and of placental origin argues in favor of the second hypothesis. However, in order to demonstrate a link between cerebrovascular and placental damage, we conducted a correlation study in subjects in the “Control” group on the one hand and in individuals in the “FAS/pFAS” group on the other (FIG. 11).

The results show that in the “Control” group, increased placental vascularization does not impact the radial organization of cortical vessels (R² 0.4719). In contrast, the placental vascularization defect observed in the “FAS/pFAS” group is closely correlated with random orientation of cortical vessels (R² 0.9995). There is therefore a very significant interaction between placental and cerebral vascular alterations.

Demonstration of a Functional Link Between Placental PlGF and its Brain Receptor

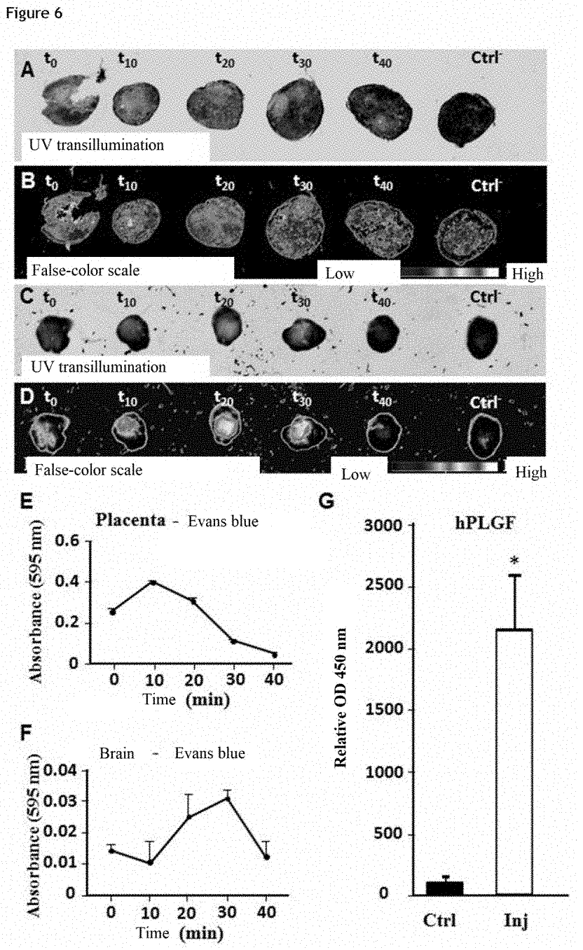

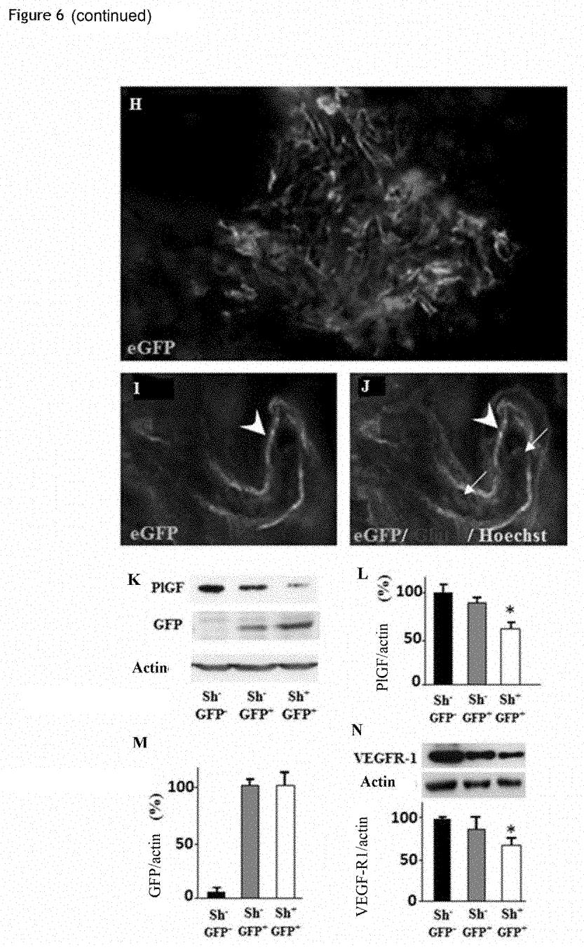

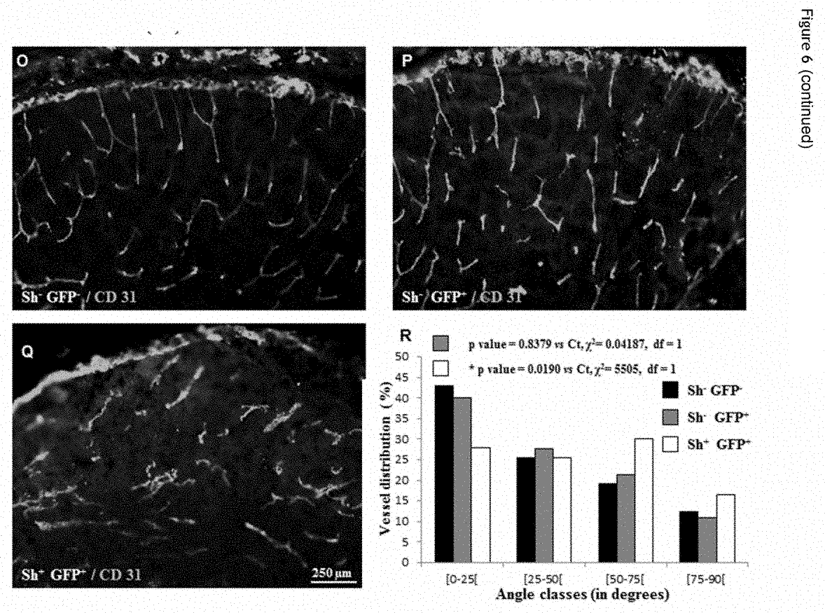

In utero administration of a fluorescent molecule to the placenta in pregnant mice (GD15) is found after 20-30 min in the fetal brain (FIG. 6). In addition, recombinant human PlGF injected into mouse placenta is detected after 30 min by ELISA in the fetal brain (FIG. 6). These data indicate that placental molecules, notably PlGF, are able to reach the fetal brain.

Invalidation by in utero placental transfection of murine PlGF by shRNA results in suppression of placental PlGF protein levels after 48 hours (FIG. 6). This effect is associated at the brain level by a drop in VEGF-R1 protein levels (FIG. 6). These results indicate that i) the specific suppression of placental PlGF directly impacts brain receptor expression, ii) the specific suppression of placental PlGF mimics the effects of alcohol on brain VEGF-R1 expression (FIGS. 2 and 6).

The expression levels of proteins known to be either involved in angiogenesis or specific to the vasculature were quantified by western blot. This work was performed in animals (mice; placenta/brain) and in humans (placenta).

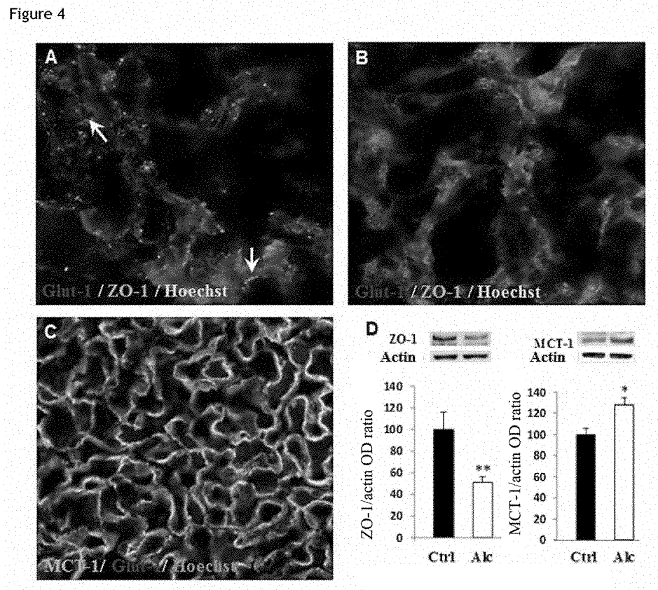

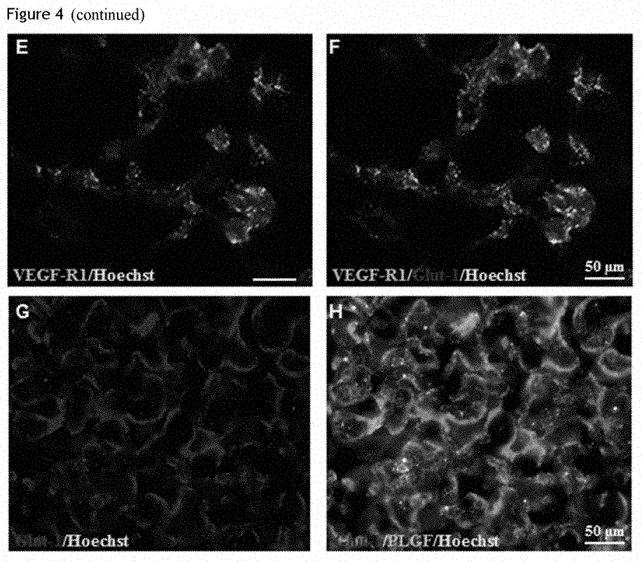

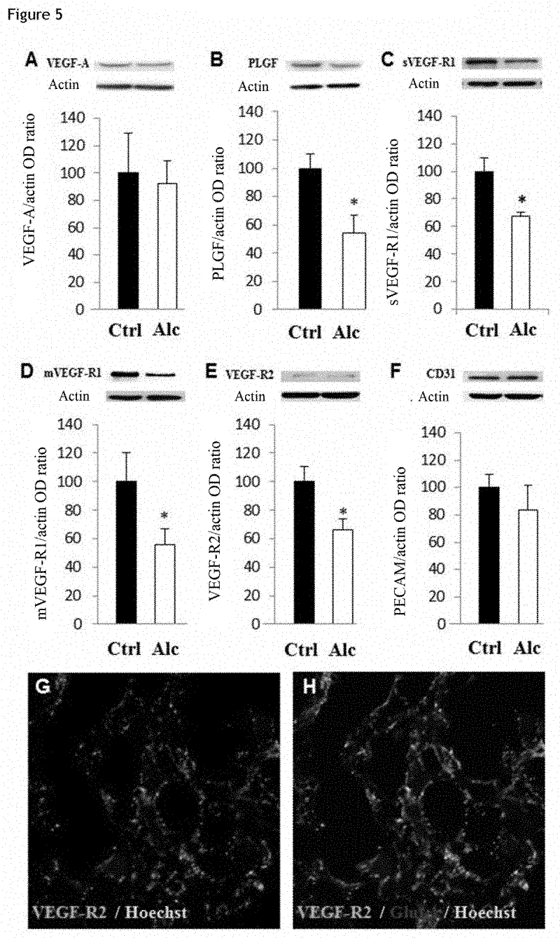

In mice, the quantification of placental VEGF_{A} and PlGF expression rates shows a significant decrease only in PlGF (for which the placenta is the only source in the body; FIG. 5). At the same time, the quantification of VEGF_{A} and PlGF receptors indicates that VEGFR1 expression (the only PlGF receptor) is decreased in both the placenta and the brain (FIGS. 2 and 5). This very marked decrease is about 50%. For its part, VEGFR2 expression in the brain is not affected. In addition, the quantification of vascular protein ZO-1, involved in the establishment of the placental and blood-brain barrier, is strongly decreased in the placenta (FIG. 4).

In parallel with the work carried out in mice, protein expression analysis was carried out on human placentas with proven maternal alcohol exposure and living children. We collected 7 “Control” and 6 “Alcohol” placentas and quantified by western blot the candidate markers identified in mice. The results indicate that in the “Alcohol” group, the expressions of PlGF and ZO-1 are very strongly decreased as in mice (FIG. 10). These data indicate that the effects of fetal alcohol exposure observed at the placental and cerebral levels are found in two different species, mice and humans.

### Examples B: Therapeutic Effect of PlGF

These examples correspond to the results obtained by tests of PlGF overexpression at the placental level thus reversing the deleterious effect of alcohol on fetal morphology and size as well as on brain angiogenesis.

1. Materials and Methods

Placental overexpression of PlGF by in vivo activation of the PGF gene in a CRISPR-dCas9 system

Combined with in vivo electroporation, the CRISPR-dCas9 approach is an innovative gene overexpression method for identifying the role of endogenous proteins in developmental processes. The PGF CRISPR-dCas9 (sc-422211-ACT) activation plasmids constituting a synergistic activation mediator (SAM) complex were designed and supplied by Santa Cruz Biotechnology. SAM binds to a specific site upstream of the PGF gene transcription initiation site, thus activating endogenous transcription of the target gene. In practice, the PGF-CRISPR dCas9 activation plasmids are transfected by in utero electroporation at GD13 in two groups of mice (“Control” and “Alcohol”). Alcohol exposure occurs between GD15 and GD20. A two-day delay between transfection of PGF CRISPR-dCas9 activation plasmids and alcohol treatment is required to allow plasmid expression and PLGF overexpression. For a given pregnant mouse, 3 placentas were transfected with the PGF CRISPR-dCas9 activation plasmids, 3 placentas were transfected with the CRISPR-cas9 (sc-418922) control plasmids targeting a 20-nt nonspecific guide RNA (negative control). The other placentas are not transfected and are used as controls (“Control” group).

2. Results

Placental Overexpression of the PGF Gene Restores Fetal Brain Angiogenesis Altered by in Utero Alcohol Exposure.

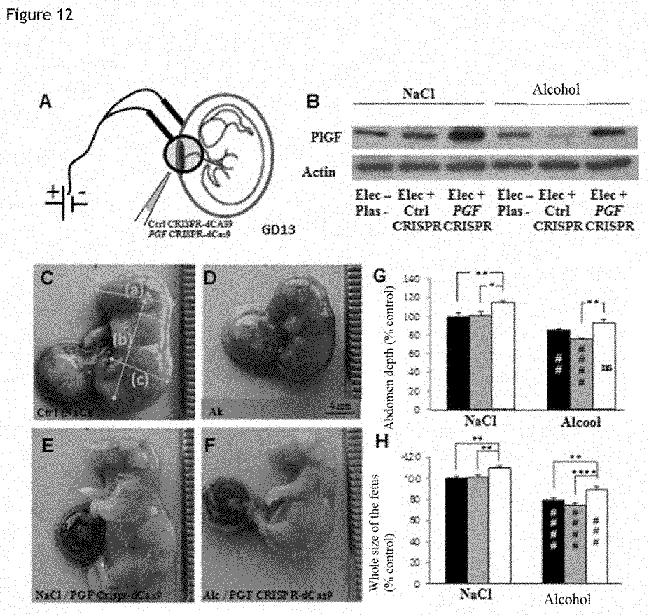

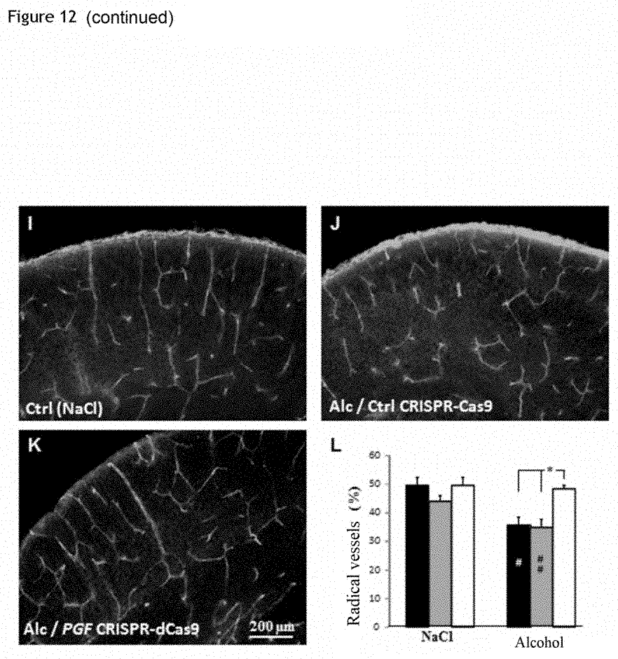

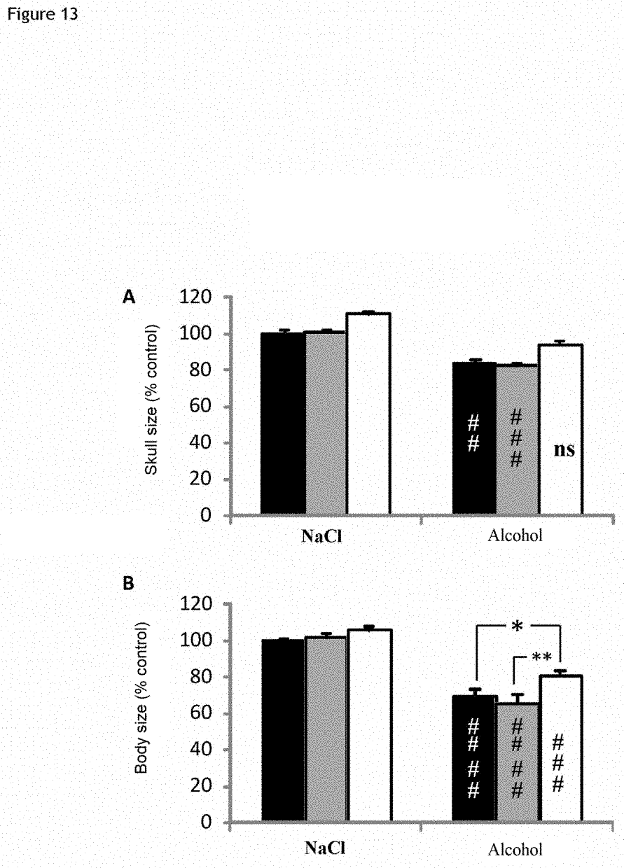

A CRISPR-dCas9 activation strategy coupled with in utero transfection was used to induce strong expression of the endogenous PGF gene in the placenta of pregnant mice not treated with alcohol (control group) and pregnant mice exposed to alcohol (alcohol group) (FIG. 12, A and B). At GD20, it was observed that in utero alcohol exposure leads to decreased intrauterine growth of the fetus (FIG. 12, C and D) with a significant decrease in head size (p<0.01; FIG. 13), body size (p<0.0001; Fig. S2), abdomen size (p<##; FIG. 12, G) and whole fetal size (p<####; FIG. 12, H). In the “Control” group, PGF overexpression induces a macromorphic evolution of the fetus (FIG. 12, C and E), with a significant increase in abdomen size (p<0.01; FIG. 12, G) and whole fetal size (p<0.01; FIG. 12, H). In the “Alcohol” group, PGF overexpression significantly increases body size (p<0.05; FIG. 13) and whole fetal size (p<0.01; FIG. 12, H). Compared with the “Control” group, PGF overexpression suppressed the deleterious effects of alcohol on head size (FIG. 13) and abdomen size (FIG. 12, G) and reduced the effects of alcohol on body size (FIG. 13) and whole fetal size (FIG. 12, H) by 38.6±2.8% and 46.8±2.9%, respectively. No effects on fetal morphology were observed in pregnant mice transfected with the CRISPR-cas9 control plasmid (FIG. 12, G and H). As previously demonstrated, in the fetal brain of E20 mice, in utero alcohol exposure leads to disorganization of the cortical vasculature (FIG. 1, A and B). Transfection of the placenta with a CRISPR-cas9 control plasmid has no effect on angiogenic alterations induced by in utero alcohol exposure (FIG. 12, I, J and L). In contrast, in pregnant mice whose placenta was transfected with an CRISPR-PGF dCas9 activation plasmid, radial organization of cortical microvessels was significantly restored (p<0.05; FIG. 4, K, L). These data represent the first demonstration that PLGF overexpression in the placenta partially or fully restores morphological deficiencies and alterations in brain angiogenesis induced by in utero alcohol exposure.

Conclusion

In view of the different results obtained by the inventors in pregnant mice, it appears that:
overexpression of PlGF in the placenta of an alcohol-exposed pregnant mouse completely eliminates alcohol exposure-related morphometric abnormalities in the abdomen and skull of the fetus. Thus, skull and abdomen size, which is reduced following alcohol exposure, returns to normal after overexpression of PlGF,
overexpression of PlGF in the placenta of an alcohol-exposed pregnant mouse reduces alcohol exposure-related morphometric abnormalities of the body of the fetus and of the fetus as a whole. Thus, the size of the fetus' body and the size of the whole fetus, reduced upon alcohol exposure, return to almost normal after overexpression of PlGF,
overexpression of PlGF at the placental level improves brain angiogenesis of the fetus altered by alcohol exposure of a pregnant mouse. The neuronal function of this fetus will thus improve,
overexpression of PlGF can be used effectively as a medicinal product in the treatment of FASD-type abnormalities, in particular to improve brain angiogenesis and/or restore the normal morphological appearance of vascularization of the fetus with FASD.

1. Placental growth factor (PlGF) for use in the prevention and/or treatment of fetal alcohol spectrum disorders (FASD) in a subject who has been exposed to alcohol in utero.

2. PlGF for use according to claim 1, characterized in that the FASD is related to cerebrovascular damage.

3. PlGF for use according to claim 1 or claim 2, characterized in that said PlGF stimulates brain angiogenesis.

4. PlGF for use according to claim 1, characterized in that the FASD is fetal alcohol syndrome (FAS), particularly when it manifests as hypotrophy in a subject who has been exposed to alcohol in utero.

5. PlGF for use according to claim 4, characterized in that the hypotrophy is a hypotrophy of the whole subject or of one of its parts selected from the torso, the abdomen and the skull.

6. PlGF for use according to any one of claims 1 to 5, characterized in that said PlGF has the sequence represented by one among SEQ ID NO: 1 to SEQ ID NO: 4.

7. PlGF for use according to any one of claims 1 to 6, characterized in that it is obtained by genetic engineering or by chemical synthesis.

8. PLGF for use according to any one of claims 1 to 7, characterized in that the subject who has been exposed to alcohol in utero is selected from an embryo, a fetus and a child, in particular a preterm child.

9. PLGF for use according to any one of claims 1 to 8, characterized in that it is administered in utero or ex utero or in utero then ex utero.

10. PlGF for use according to any one of claims 1 to 9, characterized in that it is administered ex utero to a preterm child.

11. Pharmaceutical composition for use in the prevention and/or treatment of fetal alcohol spectrum disorders (FASD) comprising a PlGF as defined in any one of claims 1 to 10 and a pharmaceutically acceptable carrier.

12. PlGF for use according to any one of claims 1 to 10 or pharmaceutical composition for use according to claim 11, said use comprising a preliminary step of identifying the subject, said identification comprising the following steps:
a) measurement of the amount of PlGF in a biological sample from said subject, preferably from the placenta or from umbilical cord blood;
b) comparison of the amount of PlGF in step a) with a reference that is a measure of the amount of PlGF in a healthy subject, and
c) determination of an FASD or of a risk of developing an FASD in said subject.

13. PlGF or composition for use according to claim 12, characterized in that the subject suffers from FASD or has been identified at risk of developing an FASD if the measured amount of PlGF in step a) is less than the reference in step b).

14. PlGF or composition for use according to claim 12, characterized in that the amount of PlGF is determined by measuring the amount of PlGF nucleic acid or the amount of the PlGF polypeptide.

15. PlGF or composition for use according to any one of claims 12 to 14, characterized in that the amount of PlGF is measured by a method selected from northern blot, Southern blot, PCR, RT-PCR, quantitative RT-PCR, SAGE and derivatives thereof, nucleic acid chips, particularly cDNA chips, oligonucleotide chips and mRNA chips, tissue chips and RNA-Seq and/or by a method selected from immunohistology, immunoprecipitation, western blot, dot blot, ELISA or ELISPOT, protein chips, antibody chips, or tissue chips coupled to immunohistochemistry, FRET or BRET techniques, microscopy or histochemistry methods, including in particular confocal and electron microscopy methods, methods based on the use of one or more excitation wavelengths and an appropriate optical method, such as an electrochemical method (voltammetric and amperometric techniques), atomic force microscopy, and radiofrequency methods, such as multipolar, confocal and non-confocal resonance spectroscopy, detection of fluorescence, luminescence, chemiluminescence, absorbance, reflectance, transmittance, and birefringence or refractive index (for example, by surface plasmon resonance, by ellipsometry, by resonant mirror method, etc.), flow cytometry, radioisotope or magnetic resonance imaging, polyacrylamide gel electrophoresis analysis (SDS-PAGE); by HPLC-mass spectrophotometry, by liquid chromatography/mass spectrophotometry/mass spectrometry (LC-MS/MS).
